SEQUENCE LISTING (8 sequences)

SEQ ID NO: 1 - protein, 58 aa, synthetic construct
IAAQHDKDQQAAFYEILHLPNLTEEQRNAFIQSLRDDPSVSLEILAEAKKLNDAQAPK

SEQ ID NO: 2 - protein, 114 aa, synthetic construct
AQHDKIQQAAFHEILHLPNLTEEQRNKFIQSLRDDPSVSLEILCEAKKLNDAQAPKIAAQHDKIQQAAFHEILHLPNLTEEQRNKFIQSLRDDPSVSLEILAEAKKLNDAQAPK

SEQ ID NO: 3 - protein, 58 aa, synthetic construct
IAAQHDKDQQAAFYEIHHLPNLTEEQRNAFIQSLRDDPSVSLCILAEAKKLNDAQAPK

SEQ ID NO: 4 - protein, 58 aa, synthetic construct
IAAQHDKDQQAAFYEHLHLPNLTEEQRNAFIQSLRDDPSVSLCILAEAKKLNDAQAPK

SEQ ID NO: 5 - protein, 58 aa, synthetic construct
IAAQHDKDQQAAFHEILHLPNLTEEQRNAFIQSLRDDPSVSLCILAEAKKLNDAQAPK

SEQ ID NO: 6 - protein, 58 aa, synthetic construct
IAAQHDKDQHAAFYEILHLPNLTEEQRNAFIQSLRDDPSVSLCILAEAKKLNDAQAPK

SEQ ID NO: 7 - protein, 58 aa, synthetic construct
IAAQHDKDQDAAFYEILHLPNLTEEQRNAFIQSLRDDPSVSLCILAEAKKLNDAQAPK

SEQ ID NO: 8 - protein, 58 aa, synthetic construct; identical to SEQ ID NO: 1

(19) United States
(12) Patent Application Publication
(10) Pub. No.: US 2025/0367575 A1
(43) Pub. Date: Dec. 4, 2025

(54) PROTEIN A CHROMATOGRAPHY VIRAL CLEARANCE AT ELUTION GREATER THAN pH 4

(71) Applicant: Purolite LLC, King of Prussia, PA (US)

(72) Inventors: Mark Hicks, St Mellons (GB); Johanna Louise Howson, Creigiau (GB); Benjamin David Summers, Pontyclun (GB); Andrew Masters, Shenley (GB)

(21) Appl. No.: 19/222,390

(22) Filed: May 29, 2025

Related U.S. Application Data
(60) Provisional application No. 63/654,653, filed on May 31, 2024.

(51) Int. Cl. B01D 15/38; B01D 15/20; B01D 15/42; C07K 1/22

(52) U.S. Cl. CPC B01D 15/3809; B01D 15/20; B01D 15/424; C07K 1/22

(57) ABSTRACT

Described herein are methods for viral clearance of samples comprising proteins using affinity chromatography. Methods include contacting an affinity chromatography resin with a liquid sample comprising protein and virus, or suspected of having virus, wherein the affinity chromatography resin comprises a cross-linked polysaccharide with a Protein A functional group and the protein comprises an Ig sequence. Methods include eluting the protein from the affinity chromatography resin with an elution liquid having a pH of about 4 to about 5.5 to produce an eluted sample, whereby the eluted sample has a reduction in virus compared to the liquid sample.

### CROSS REFERENCE TO RELATED APPLICATIONS

This application claims priority under 35 U.S.C. § 119 to provisional patent application U.S. Ser. No. 63/654,653, filed May 31, 2024. The provisional patent application is herein incorporated by reference in its entirety.

### INCORPORATION OF SEQUENCE LISTING

The sequence listing contained in the file named “P14761US02.XML” which is 8,406 bytes (measured in MS-Windows®), comprises 7 biological sequences, and was created on May 23, 2025, is electronically filed herewith and is incorporated herein by reference in its entirety.

### TECHNICAL FIELD

The disclosure relates to the field of liquid chromatography, and more specifically relates to methods of viral clearance by affinity chromatography including contacting an affinity chromatography resin with a liquid sample comprising protein and virus, or suspected of having virus, wherein the affinity chromatography resin comprises a cross-linked polysaccharide with an Ig binding protein or an Ig binding domain as a functional group and the protein comprises an Ig sequence. Methods include eluting the protein from the affinity chromatography resin with an elution liquid having a pH of about 4 to about 5.5 to produce an eluted sample, whereby the eluted sample has a reduction in virus compared to the liquid sample.

### BACKGROUND

Manufacturing biologics includes validation of manufacturing processes to ensure biologics safety. Included in the process validation is reducing viral contamination of the manufactured biologics. Methods of viral clearance include capture chromatography, wherein target proteins are captured and virus flows through, thereby clearing virus from a protein sample. Other methods include inactivation of viruses by incubating the biologics at low pH and by filtration.

Methods of viral clearance include liquid chromatography, such as ion exchange chromatography and affinity chromatography. In ion exchange chromatography, molecules are separated according to the strength of their overall ionic interaction with a stationary phase. Affinity chromatography is used to isolate and purify biomolecules such as proteins and includes, for example, monoclonal antibody purification. In affinity chromatography, a stationary phase is modified with ligands that bind the target proteins to the stationary phase.

Affinity chromatography using Protein A as a ligand can be used to capture monoclonal antibodies (mAb) and molecules that possess an Fc-domain, such as fusion proteins and biospecific antibodies. However, conditions for eluting bound proteins include a low pH (<4.0) that can cause protein degradation (e.g. fragmentation, aggregation) for molecules sensitive to low pH.

Therefore, there is a need in the art for improved methods of purification of proteins that possess an Fc-domain using affinity chromatography that increases viral clearance and results in eluted proteins that are undamaged by low pH elution conditions.

It is therefore an object of this disclosure to provide improved methods for viral clearance using affinity chromatography.

Other objects, embodiments and advantages of this disclosure will be apparent to one skilled in the art in view of the following disclosure, the drawings, and the appended claims.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

According to some aspects of the present disclosure, methods of viral clearance by affinity chromatography comprise: providing a liquid sample comprising protein and virus, wherein the protein comprises an Ig sequence; providing an affinity chromatography resin comprising at least one Ig binding protein coupled to thereto; contacting the affinity chromatography resin with the liquid sample under conditions that allow binding of the protein comprising the Ig sequence to the at least one Ig binding protein; and eluting the protein from the affinity chromatography resin with an elution liquid having a pH of about 4 to about 5.5, or preferably greater than 4.5, to produce an eluted protein sample; wherein the eluted protein sample has a reduction in virus compared to the liquid sample.

According to further aspects of the present disclosure, methods of viral clearance by affinity chromatography can comprise: providing a liquid sample comprising protein and virus, wherein the protein comprises an Ig sequence; providing an affinity chromatography resin comprising at least one Ig binding protein coupled to thereto; contacting the affinity chromatography resin with the liquid sample under conditions that allow binding of the protein comprising the Ig sequence to the at least one Ig binding protein; washing the affinity chromatography resin with at least one wash buffer; and eluting the protein from the affinity chromatography resin with an elution liquid having a pH of about 4 to about 5.5 to produce an eluted protein sample; wherein the eluted protein sample has a reduction in virus compared to the liquid sample.

According to further aspects of the present disclosure, methods of viral clearance by affinity chromatography, comprise: providing a liquid sample comprising protein and virus, wherein the protein comprises an Ig sequence; providing an affinity chromatography resin comprising at least one Ig binding protein coupled to thereto; contacting the affinity chromatography resin with the liquid sample under conditions that allow binding of the protein comprising the Ig sequence to the at least one Ig binding protein; washing the affinity chromatography resin with at least one wash buffer, wherein the wash buffer has a pH of about 6.0 to about 10.0 with a salt concentration of about 50 mM to about 1000 mM; and eluting the protein from the affinity chromatography resin with an elution liquid having a pH of about 4 to about 5.5 to produce an eluted protein sample; wherein the eluted protein sample has a reduction in virus compared to the liquid sample.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIGS. 1A-1B are chromatogram profiles run in duplicate of harvested cell culture fluid (HCCF) comprising IgG4 class antibody run through an affinity chromatography resin of Resin 1 (Example 1) with an elution buffer at pH 3.5. FIG. 1A is a first run with HCCF. FIG. 1B is a second run with HCCF using a separate resin and HCCF sample from FIG. 1A.

FIGS. 2A-2B are chromatogram profiles run in duplicate of harvested cell culture fluid (HCCF) comprising IgG4 class antibody run through an affinity chromatography resin, Mab Select SuRe LX. FIG. 2A is a first run with HCCF. FIG. 2B is a second run with HCCF using a separate resin and HCCF sample from FIG. 2A.

FIGS. 3A-3B are chromatogram profiles run in duplicate of harvested cell culture fluid (HCCF) comprising IgG4 class antibody run through an affinity chromatography resin of Resin 2 as a comparative example to Resin 1 (Example 1). FIG. 3A is a first run with HCCF. FIG. 3B is a second run with HCCF using a separate resin and HCCF sample from FIG. 3A.

FIGS. 4A-4B are chromatogram profiles run in duplicate of harvested cell culture fluid (HCCF) comprising IgG4 class antibody run through an affinity chromatography resin of Resin 1 (Example 1) with an elution buffer at pH 4.5. FIG. 4A is a first run with HCCF. FIG. 4B is a second run with HCCF using a separate resin and HCCF sample from FIG. 4A.

FIGS. 5A-5C are chromatogram profiles of harvested cell culture fluid (HCCF) spiked with Murine Minute Virus (MMV) applied to an affinity chromatography resin of Resin 1 (Example 1) with an elution buffer at pH 3.5. FIG. 5A is a chromatogram of a first HCCF sample spiked with MMV and eluted at pH 3.5. FIG. 5B is a chromatogram of a second HCCF sample spiked with MMV and eluted at pH 3.5. FIG. 5C is an overlay of the UV 280 nm profiles from FIG. 5A and 5B showing reproducibility in duplicate.

FIGS. 6A-6C are chromatogram profiles of harvested cell culture fluid (HCCF) spiked with Xenotropic Murine Leukemia Virus (MLV) applied to an affinity chromatography resin of Resin 1 (Example 1) with an elution buffer at pH 3.5. FIG. 6A is a chromatogram of a first HCCF sample spiked with MLV and eluted at pH 3.5. FIG. 6B is a chromatogram of a second HCCF sample spiked with MLV and eluted at pH 3.5. FIG. 6C is an overlay of the UV 280 nm profiles from FIG. 6A and 6B showing reproducibility in duplicate.

FIGS. 7A-7C are chromatogram profiles of harvested cell culture fluid (HCCF) spiked with Murine Minute Virus (MMV) applied to an affinity chromatography resin of Resin 1 (Example 1) with an elution buffer at pH 4.5. FIG. 7A is a chromatogram of a first HCCF sample spiked with MMV and eluted at pH 4.5. FIG. 7B is a chromatogram of a second HCCF sample spiked with MMV and eluted at pH 4.5. FIG. 7C is an overlay of the UV 280 nm profiles from FIG. 7A and 7B showing reproducibility in duplicate.

FIGS. 8A-8C are chromatogram profiles of harvested cell culture fluid (HCCF) spiked with Xenotropic Murine Leukemia Virus (MLV) applied to an affinity chromatography resin of Resin 1 (Example 1) with an elution buffer at pH 4.5. FIG. 8A is a chromatogram of a first HCCF sample spiked with MMV and eluted at pH 4.5. FIG. 8B is a chromatogram of a second HCCF sample spiked with MMV and eluted at pH 4.5. FIG. 8C is an overlay of the UV 280 nm profiles from FIG. 8A and 8B showing reproducibility in duplicate.

FIGS. 9A-9C are chromatogram profiles of harvested cell culture fluid (HCCF) spiked with Murine Minute Virus (MMV) applied to an affinity chromatography resin of Resin 2 as a comparative example to Resin 1 (Example 1). FIG. 9A is a chromatogram of a first HCCF sample spiked with MMV. FIG. 9B is a chromatogram of a second HCCF sample spiked with MMV. FIG. 9C is an overlay of the UV 280 nm profiles from FIG. 9A and 9B showing reproducibility in duplicate.

FIGS. 10A-10C are chromatogram profiles of harvested cell culture fluid (HCCF) spiked with Xenotropic Murine Leukemia Virus (MLV) applied to an affinity chromatography resin of Resin 2 as a comparative example to Resin 1 (Example 1). FIG. 10A is a chromatogram of a first HCCF sample spiked with MMV. FIG. 10B is a chromatogram of a second HCCF sample spiked with MMV. FIG. 10C is an overlay of the UV 280 nm profiles from FIG. 10A and 10B showing reproducibility in duplicate.

FIGS. 11A-11C are chromatogram profiles of harvested cell culture fluid (HCCF) spiked with Murine Minute Virus (MMV) applied to a Protein A chromatography resin, Mab Select SuRe LX. FIG. 11A is a chromatogram of a first HCCF sample spiked with MMV. FIG. 11B is a chromatogram of a second HCCF sample spiked with MMV. FIG. 11C is an overlay of the UV 280 nm profiles from FIG. 11A and 11B showing reproducibility in duplicate.

FIGS. 12A-12C are chromatogram profiles of harvested cell culture fluid (HCCF) spiked with Xenotropic Murine Leukemia Virus (MLV) applied to a Protein A chromatography resin, Mab Select SuRe LX. FIG. 12A is a chromatogram of a first HCCF sample spiked with MMV. FIG. 12B is a chromatogram of a second HCCF sample spiked with MMV. FIG. 12C is an overlay of the UV 280 nm profiles from FIG. 12A and 12B showing reproducibility in duplicate.

Various embodiments of the present disclosure will be described in detail with reference to the drawings, wherein like reference numerals represent like parts throughout the several views. Reference to various embodiments does not limit the scope of the disclosure. Figures represented herein are not limitations to the various embodiments according to the disclosure and are presented for exemplary illustration of the invention. An artisan of ordinary skill in the art need not view, within isolated figure(s), the near infinite number of distinct permutations of features described in the following detailed description to facilitate an understanding of the present invention.

### DETAILED DESCRIPTION

The present disclosure is not to be limited to that described herein, such as particular methodologies, protocols, and reagents as described, as these may vary and are understood by skilled artisans. No features shown or described are essential to permit basic operation of the present disclosure unless otherwise indicated. It has been beneficially found that viral clearance of a liquid sample comprising virus and protein comprising an Ig sequence, such monoclonal antibodies (mAb) and molecules that possess an Fc-domain, such as fusion proteins and bispecific antibodies, can be improved by affinity chromatography using a modified cross-linked agarose having an Ig binding domain or Ig binding protein and elution at a pH of about 4 to 5.5 compared to methods of elution at lower pH of 3.5.

It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. This applies regardless of the breadth of the range.

All publications, including all patents, patent applications and other patent and non-patent publications cited or mentioned herein are incorporated herein by reference for at least the purposes that they are cited; including for example, for the disclosure or descriptions of methods of materials which may be used. Nothing herein is to be construed as an admission that a publication or other reference (including any reference cited in the Background section) is prior art to the invention or that the invention is not entitled to antedate such disclosure, for example, by virtue of prior invention.

### Definitions

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

As used herein, the term “Ig binding protein” or “immunoglobulin-binding protein” is used to describe proteins that are capable of specifically binding to an immunoglobulin. Further, as used herein, the term “Ig binding domain” or “immunoglobulin-binding domain” is used to describe proteins that are capable of specifically binding to an immunoglobulin. The Ig binding proteins or Ig binding domains of the present invention are sometimes referred to herein as ligands of the invention. The “immunoglobulin” or “Ig” as understood herein can include, but is not necessarily limited to, mammalian IgG, such as for example human IgG1, human IgG2, human IgG4, mouse IgG, rat IgG, goat IgG, bovine IgG, guinea pig IgG, rabbit IgG; human IgM, human IgA; and an immunoglobulin fragment comprising a Fc region (also referred to as “Fc fragment” or “Fc”) and/or an immunoglobulin fragment comprising a Fab region (also referred to as “Fab fragment” or “Fab”). The Ig binding proteins are capable of binding to entire immunoglobulins, and to Ig fragments comprising a Fc region and/or Ig fragments comprising a Fab region. The definition “immunoglobulin” as understood herein includes fusion proteins comprising an immunoglobulin, fragment of an immunoglobulin comprising a Fc region (Fc fragment), fragment of an immunoglobulin comprising a Fab region (Fab fragment), fusion proteins comprising a fragment of an immunoglobulin comprising a Fc region, fusion proteins comprising a fragment of an immunoglobulin comprising a Fab region, conjugates comprising an Ig or an Ig fragment comprising a Fc region (Fc fragment), and conjugates comprising an Ig fragment comprising a Fab region (Fab fragment).

As will be appreciated by a person of ordinary skill in the art, the terms “immunoglobulin” and “antibody” may be used interchangeably herein. Any definitions disclosed herein concerning the term “immunoglobulin” apply to the term “antibody” accordingly.

The term “binding” according to the invention preferably relates to a specific binding. “Specific binding” means that an Ig binding protein or an Ig binding domain binds stronger to an immunoglobulin for which it is specific compared to the binding to another non-immunoglobulin target.

The term “binding activity” refers to the ability of an Ig binding protein or Ig binding domain of the invention to bind to immunoglobulin. For example, the binding activity can be determined before and/or after alkaline treatment. The terms (immunoglobulin) “binding activity” and “binding capacity” may be used interchangeably herein. The binding activity can be determined for an Ig binding protein or for an Ig binding protein coupled to a matrix, i.e., for an immobilized Ig binding protein. Also, the binding activity can be determined for an Ig binding domain or for an Ig binding domain coupled to a matrix, i.e., for an immobilized Ig binding domain. The term “artificial” refers to an object that is not naturally occurring, i.e. the term refers to an object that has been produced or modified by man. For example, a polypeptide or polynucleotide sequence that has been generated by man (e.g. for example in a laboratory by genetic engineering, by shuffling methods, or by chemical reactions, etc.) or intentionally modified is artificial.

The methods of the present disclosure may comprise, consist essentially of, or consist of the components and steps described as well as other components and steps described herein. As used herein, the word “comprise”, and variations such as “comprises” and “comprising”, will be understood to imply the inclusion of a stated member, integer or step or group of members, integers or steps but not the exclusion of any other member, integer or step or group of members, integers or steps. As used herein, “consisting essentially of” means that the methods may include additional components and steps, but only if the additional components and steps do not materially alter the basic and novel characteristics of the claimed methods.

Unless defined otherwise, all technical and scientific terms used above have the same meaning as commonly understood by one of ordinary skill in the art to which embodiments of the present disclosure pertain.

The terms “invention” or “present invention” are not intended to refer to any single embodiment of the particular invention but encompass all possible embodiments as described in the specification and the claims.

The term “about,” as used herein, refers to variation in the numerical quantity that can occur, for example, through typical measuring techniques and equipment, with respect to any quantifiable variable, including, but not limited to, concentration, mass, volume, size, time, temperature, pH, humidity, molar ratios, and the like. The term “about” also encompasses these variations. Whether or not modified by the term “about,” the claims include equivalents to the quantities.

As used herein, the term “between” is inclusive of any endpoints noted relative to a described range.

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

As used herein, the term “exemplary” refers to an example, an instance, or an illustration, and does not indicate a most preferred embodiment unless otherwise stated.

The term “generally” encompasses both “about” and “substantially.”

As used herein the term “polymer” refers to a molecular complex comprised of a more than ten monomeric units and generally includes, but is not limited to, homopolymers, copolymers, such as for example, block, graft, random and alternating copolymers, terpolymers, and higher “x”mers, further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

The terms “protein” and “polypeptide” refer to any linear molecular chain of two or more amino acids linked by peptide bonds and does not refer to a specific length of the product. Thus, “peptides”, “protein”, “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-translational modifications of the polypeptide, including without limitation glycosylation, acetylation, phosphorylation, amidation, proteolytic cleavage, modification by non-naturally occurring amino acids and similar modifications which are well-known in the art. Thus, Ig binding proteins comprising two or more protein domains also fall under the definition of the term “protein” or “polypeptides”.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

The term “substantially” refers to a great or significant extent. “Substantially” can thus refer to a plurality, majority, and/or a supermajority of said quantifiable variable, given proper context.

### Viral Clearance

For monoclonal antibodies (mAbs) produced in mammalian cells and other Fc containing protein biologics, viral safety is a potential risk. Regulatory agencies require the efficacy of downstream manufacturing process at removing or inactivating virus to be validated. Generally, viral reduction through the downstream process is achieved by column chromatography and dedicated viral clearance steps (i.e., low pH/detergent treatment and nanofiltration) to ensure robust clearance (removal or inactivation) of potential endogenous and adventitious viruses. In general, Protein A and polishing chromatography steps all can provide some level of virus removal.

### Viral Clearance Methods

Virus removal capacities by chromatography are determined using scale-down models mimicking the corresponding full-scale manufacturing process. Such viral clearance validation processes involve spiking virus in the chromatography column load (i.e. liquid sample or input sample) and calculating viral reduction by comparing the amount of virus in the load to that in the processed sample. Viral clearance is typically expressed as log reduction values (LRVs) and the added values of all unit operations in the manufacturing process represent the viral clearance capacity of the entire purification process. Viral clearance studies are often conducted with Xenotropic Murine Leukemia Virus (MLV) and Murine Minute Virus (MMV).

Downstream processing of Mab or other proteins containing Fc sequences include chromatography which removes virus by binding affinity, charge, or hydrophobicity. Chromatography methods used for viral clearance include Protein A, anion exchange, cation exchange, hydrophobic interaction, and mixed-mode chromatography (both ionic and hydrophobic function groups bound to the resin). Regardless of the technology, biomanufacturers must demonstrate the clearance capabilities of different downstream steps as part of the viral safety assessment process.

### Protein A Chromatography

Protein A chromatography has been used for capture and initial purification of mAbs. Despite Protein A's high selectivity toward mAbs, this step typically only achieves modest levels of viral clearance (mean LRVs for MLV and MMV are 2.98 and 2.32, respectively) and the exact level of reduction varies significantly among different mAbs. Interactions between the mAb and the virus may contribute to retention of the virus on the resin.

Affinity chromatography resins comprising at least one Ig binding protein coupled to thereto can include for example those disclosed in U.S. Patent Application Publication No. US 2023/0295223, which is incorporated herein by reference in its entirety. Affinity chromatography resins can include Ig binding proteins as an affinity ligand, such as Protein A, that are modified for stability at high concentrations of NaOH and provide for the elution of antibodies (immunoglobulins) from the affinity ligand at pH between 3.7 and higher, such as pH 4.3 and above, for example, up to pH 5.5. Modifications of Protein A can include mutagenesis of one or more amino acid residues and/or multimerization.

Affinity chromatography resins comprising at least one Ig binding protein coupled to thereto can include an affinity chromatography resin with uniform agarose beads that are highly cross-linked and modified with a Protein A functional group.

The affinity chromatography resin of Resin 1 (Example 1) provides elution of pH-sensitive mAbs and Fc-containing proteins, which can become unstable at low pH levels typically used for elution with other protein A resins (e.g. pH 3-3.5). The Resin 1 provides alkaline stability (maintains capacity after exposure to 0.1 M NaOH for 120 hours), capacity of 60 g/L for polyclonal human IgG, and elution up to about pH 5.5.

### Methods

Methods of viral clearance using affinity chromatography resin of Resin 1 were found to reduce viral load in the eluted protein samples to a greater degree when elution conditions were at pH 4.5 compared to pH 3.5, as shown in Table 1.

TABLE 1
Log Reduction Factors (Log₁₀).
Log Reduction Factors (Log₁₀)

| | Run 1 | Run 2 |
|---|---|---|
| Resin 1 - pH 3.5 | | |
| MLV | 3.71 ± 0.06 | 3.76 ± 0.06 |
| MMV | 2.99 ± 0.46 | 2.85 ± 0.53 |
| Resin 1 - pH 4.5 | | |
| MLV | 4.85 ± 0.06 | 5.02 ± 0.06 |
| MMV | 3.85 ± 0.52 | 4.27 ± 0.43 |

### Liquid Sample

As described herein, methods of viral clearance by affinity chromatography can comprise contacting the affinity chromatography resin with a liquid sample comprising protein and virus. In some cases, the liquid sample can be suspected of containing virus. In some cases, the liquid sample can be any sample with antibodies, antibody related products, Fc-fusion proteins, or any protein comprising an Fc sequence that is in need of a viral clearance purification step for regulatory purposes to ensure drug safety. For example, the liquid sample can be a cell lysate, cell media containing proteins secreted from cells, blood, plasma, or lymphatic fluid.

### Contacting Sample to Affinity Chromatography Resin

In some embodiments, the liquid sample can be prepared for contact with the affinity chromatography resin by adjusting the liquid sample's protein concentration as appropriate for the Ig binding capacity of the matrix. The binding capacity or binding affinity for immunoglobulin of the Ig binding protein or Ig binding domain of the affinity chromatography resin can be readily determined by a skilled person and is information provided with commercially available chromatography resins. For example, the affinity chromatography resin of Resin 1 has a capacity of 60 g/L for polyclonal human IgG.

In some embodiments, the liquid sample can be filtered prior to contacting the affinity chromatography resin. For example, the liquid sample can be filtered through a 0.22 μm polyethersulfone (PES) membrane. The filtration step can sterilize the liquid sample and/or remove larger impurities that can impede flow through the matrix.

### Washing

In some embodiments, the method of viral clearance comprising affinity chromatography may further comprise one or more washing steps under conditions sufficient to remove from the affinity chromatography resin some or all molecules that are non-specifically bound thereto. Non-specifically bound means any binding that does not involve an interaction between the at least one Ig binding protein or Ig binding domain and an Ig.

The washing steps may include washing the affinity chromatography resin with at least one wash buffer with a pH of about pH 6.0 to about pH 10.0. In some embodiments, the step of washing the affinity chromatography resin with the at least one wash buffer comprises contacting the affinity chromatography resin with a sequential series of wash buffers. For example, the series of wash buffers can include a first wash buffer having a pH of about 7.4, then a second wash buffer having a pH of about 8.0, and then a third wash buffer having a pH of about 7.0.

In some embodiments, the at least one wash buffer can include one or more salts. For example, the at least one wash buffer can include salts such as sodium chloride and/or sodium phosphate. The concentration of salts in the at least one wash buffer can be any concentration effective for disrupting and washing away proteins or other molecules that nonspecifically bind or interact with the affinity chromatography resin. In some embodiments, the at least one wash buffer may include salt concentrations of about 50 mM to about 1000 mM. In an example of washing steps, the first wash buffer can comprise sodium phosphate and sodium chloride having a pH of about 7.4, the second wash buffer can comprise Tris buffer and sodium chloride having a pH of about 8.0, and the third wash buffer can comprise sodium phosphate having a pH of about 7.0.

### Elution

In some embodiments, the elution of the immunoglobulin from the affinity chromatography resin can be affected through a change in pH. In some embodiments, the step of eluting the protein from the affinity chromatography resin comprises elution with an elution liquid having a pH of about 4 to about 5.5 to produce an eluted protein sample, wherein the eluted protein sample has a reduction in virus compared to the liquid sample.

In some embodiments, the elution liquid is a buffer comprising sodium acetate.

Elution of the affinity chromatography resin with the elution liquid may provide elution of at least about 95%, at least about 96%, at least about 97%, at least about 98%, at least about 99%, or 100% of Ig containing proteins at a pH of greater than or equal to pH 4.0 (e.g., about pH 4.0, about pH 4.5, about pH 5.0, or about pH 5.5). In various embodiments, the pH of the elution liquid is preferably greater than 4.5. Due to the high stability of the Ig binding proteins and Ig binding domains in the affinity chromatography resin, elution liquids with greater than or equal to pH 4.0 can be used for the elution of Ig proteins (see Examples).

For example, elution of the affinity chromatography resin of Resin 1 at either pH 3.5 or the milder pH 4.5 both provide effective recovery of protein comprising the Ig sequence, as shown in Table 2. However, it was found that elution at the milder pH 4.5 provided a greater virus reduction factor (log10) as compared to elution at pH 3.5 (Table 3), suggesting that the virus remained bound to the resin at the higher elution pH. As detailed in Example 2 below, for clearance of MLV, the virus log reduction for affinity chromatography resin of Resin 1 with high elution buffer pH 4.5 showed markedly higher virus removal (log reduction of 4.85±0.06 log10) compared to eluting the same resin with the lower pH elution buffer at pH 3.5 (log reduction of 3.71±0.06 log10). For clearance of MMV, the virus log reduction for affinity chromatography resin of Resin 1 with high elution buffer pH 4.5 also showed markedly higher virus removal (log reduction of 3.85±0.52 log10) compared to eluting the same resin with the lower pH elution buffer at pH 3.5 (log reduction of 2.85±0.53 log10). For both the viruses studied, performance and results for duplicate runs per virus were within 1 log of each other, indicating consistent performance of the steps and methods.

TABLE 2
% Yield of IgG Protein (chromatography runs in duplicate).

| | Yield (%) |
|---|---|
| Run Elution at pH 3.5 | |
| MLV Run1 | 109.3 |
| MLV Run 2 | 103.6 |
| MMV Run 1 | 106.3 |
| MMV Run 2 | 106.9 |
| Run Elution at pH 4.5 | |
| MLV Run1 | 102.6 |
| MLV Run 2 | 103.4 |
| MMV Run 1 | 102.2 |
| MMV Run 2 | 102.3 |

TABLE 3
Virus load, output, and overall reduction.

| | | | Reduction |
|---|---|---|---|
| | Total Load | Total output | factor |
| | (log₁₀) | (log₁₀) | (log₁₀) |
| Run Elution at pH 3.5 | | | |
| MLV Run 1 | 11.40 ± 0.04 | 7.69 ± 0.04 | 3.71 ± 0.06 |
| MLV Run 2 | 11.40 ± 0.04 | 7.64 ± 0.04 | 3.76 ± 0.06 |
| MMV Run 1 | 8.43 ± 0.33 | 5.44 ± 0.31 | 2.99 ± 0.45 |
| MMV Run 2 | 8.43 ± 0.33 | 5.58 ± 0.42 | 2.85 ± 0.53 |
| Run Elution at pH 4.5 | | | |
| MLV Run 1 | 11.39 ± 0.04 | 6.53 ± 0.04 | 4.85 ± 0.06 |
| MLV Run 2 | 11.39 ± 0.04 | 6.36 ± 0.04 | 5.02 ± 0.06 |
| MMV Run 1 | 8.25 ± 0.38 | 4.40 ± 0.36 | 3.85 ± 0.52 |
| MMV Run 2 | 8.25 ± 0.38 | 3.98 ± 0.21 | 4.27 ± 0.43 |

### EMBODIMENTS

The present disclosure is further defined by the following numbered embodiments:
1. A method of viral clearance by affinity chromatography, comprising: providing a liquid sample comprising protein and virus, wherein the protein comprises an Ig sequence; providing an affinity chromatography resin comprising at least one Ig binding protein coupled to thereto; contacting the affinity chromatography resin with the liquid sample under conditions that allow binding of the protein comprising the Ig sequence to the at least one Ig binding protein; and eluting the protein from the affinity chromatography resin with an elution liquid having a pH of about 4 to about 5.5 to produce an eluted protein sample; wherein the eluted protein sample has a reduction in virus compared to the liquid sample.
2. The method of paragraph 1, wherein the reduction in virus is at least about 3.8 log₁₀.
3. The method of paragraph 1, wherein the reduction in virus is about 3.8 log₁₀ to about 4.8 log₁₀.
4. The method of any one of paragraphs 1-3, wherein flow rate of the affinity chromatography resin is up to about 200 cm/hr.
5. The method of any one of paragraphs 1-4, wherein at least a portion of the virus in the first liquid sample remains bound to the affinity chromatography resin when the protein is eluted.
6. The method of any one of paragraphs 1-5, wherein the affinity chromatography resin comprises a cross-linked polysaccharide.
7. The method of any one of paragraphs 1-6, wherein the cross-linked polysaccharide comprises agarose.
8. The method of any one of paragraphs 6-7, wherein the cross-linked polysaccharide is a caustic resistant agarose.
9. The method of any one of paragraphs 1-8, further comprising washing the affinity chromatography resin with at least one wash buffer after the step of contacting the affinity chromatography resin with the liquid sample comprising the protein and virus and before the step of eluting the protein.
10. The method of paragraph 9, wherein the at least one wash buffer has a pH equal to or above about pH 7.0.
11. The method of any one of paragraphs 9-10, wherein the step of washing the affinity chromatography resin with the at least one wash buffer comprises contacting the affinity chromatography resin with a first wash buffer having a pH of about 7.4, then a second wash buffer having a pH of about 8.0, and then a third wash buffer having a pH of about 7.0.
12. The method of paragraph 11, wherein the first wash buffer comprises sodium phosphate and sodium chloride having a pH of about 7.4, the second wash buffer comprises Tris buffer and sodium chloride having a pH of about 8.0, and the third wash buffer comprises sodium phosphate having a pH of about 7.0.
13. The method of any one of paragraphs 1-12, wherein the elution liquid is a buffer comprising sodium acetate, citrate, and/or glycine.
14. The method of any one of paragraphs 1-13, wherein the elution liquid has a pH of about 4.5.

15. The method of any one of paragraphs 1-14, wherein the elution liquid has a pH greater than 4.5.
16. The method of any one of paragraphs 1-15, wherein the Ig binding protein comprises a cross-linked polysaccharide with a Protein A functional group.
17. The method of any one of paragraphs 1-16, wherein more than 95% of the protein comprising the Ig sequence is eluted from the affinity chromatography resin.
18. The method of any one of paragraphs 1-17, wherein the at least one Ig binding protein comprises a protein having at least 80% amino acid sequence identity to sequence SEQ ID NO: 1, wherein SEQ ID NO: 1 has at least one of the following mutations: (a) the amino acid corresponding to one or more of position 8 is isoleucine, leucine, valine or an aromatic amino acid; (b) the amino acid corresponding to position 8 is isoleucine or tyrosine; (c) the amino acid corresponding to one or more of positions 10, 14, 16, 17, 18, and 28 is/are selected from the group consisting of histidine or acidic amino acids selected from aspartate or glutamate; (d) the amino acid corresponding to position 14 is histidine; (e) the amino acid corresponding to position 17 is histidine; (f) the amino acid corresponding to position 16 is histidine; (g) the amino acid corresponding to position 10 is histidine (h) the amino acid corresponding to position 10 is asparagine; (i) the amino acid corresponding to position 8 is isoleucine, the amino acid corresponding to position 14 is histidine, and the amino acid corresponding to position 29 is lysine; and preferably the amino acid corresponding to position 46 is cysteine; (j) the amino acid corresponding to position 43 or 46 is cysteine; and/or (k) the amino acid corresponding to position 29 is lysine.
19. The method of paragraph 18, wherein the at least one Ig binding protein comprises a protein having at least 85%, 90%, 95%, or 99% amino acid sequence identity to sequence SEQ ID NO: 1 with at least one of the mutations in (a)-(k) in paragraph 18.
20. The method of any one of paragraphs 18-19, wherein the at least one Ig binding protein comprises a protein having at least 80%, preferably at least 95%, an amino acid sequence identity to sequence of SEQ ID NO: 2.
21. A method of viral clearance by affinity chromatography, comprising: providing a liquid sample comprising protein and virus, wherein the protein comprises an Ig sequence; providing an affinity chromatography resin comprising at least one Ig binding protein coupled to thereto; contacting the affinity chromatography resin with the liquid sample under conditions that allow binding of the protein comprising the Ig sequence to the at least one Ig binding protein; washing the affinity chromatography resin with at least one wash buffer; and eluting the protein from the affinity chromatography resin with an elution liquid having a pH of about 4 to about 5.5 to produce an eluted protein sample; wherein the eluted protein sample has a reduction in virus compared to the liquid sample.
22. The method of paragraph 21, wherein the step of washing the affinity chromatography resin comprises contacting the affinity chromatography resin with a first wash buffer having a pH of about 7.4, then a second wash buffer having a pH of about 8.0, and then a third wash buffer having a pH of about 7.0.
23. The method of paragraph 22, wherein the first wash buffer comprises sodium phosphate and sodium chloride having a pH of about 7.4, the second wash buffer comprises Tris buffer and sodium chloride having a pH of about 8.0, and the third wash buffer comprises sodium phosphate having a pH of about 7.0.
24. The method of any one of paragraphs 21-23, wherein the Ig binding protein comprises a cross-linked polysaccharide with a Protein A functional group.
25. The method of any one of paragraphs 21-24, wherein the reduction in virus is at least about 3.8 log₁₀.
26. The method of paragraph 25, wherein the reduction in virus is about 3.8 log₁₀ to about 4.8 log₁₀.
27. The method of any one of paragraphs 21-26, wherein at least a portion of the virus in the first liquid sample remains bound to the affinity chromatography resin when the protein is eluted.
28. The method of any one of paragraphs 21-27, wherein the affinity chromatography resin comprises a cross-linked polysaccharide.
29. The method of paragraph 28, wherein the cross-linked polysaccharide comprises agarose.
30. The method of any one of paragraphs 28-29, wherein the cross-linked polysaccharide is a caustic resistant agarose.
31. The method of any one of paragraphs 21-30, wherein the at least one Ig binding protein comprises a protein having at least 80% amino acid sequence identity to sequence SEQ ID NO: 1, wherein SEQ ID NO:1 has at least one of the following mutations: (a) the amino acid corresponding to one or more of position 8 is isoleucine, leucine, valine or an aromatic amino acid; (b) the amino acid corresponding to position 8 is isoleucine or tyrosine; (c) the amino acid corresponding one or more of positions 10, 14, 16, 17, 18, and 28 is/are selected from the group consisting of histidine or acidic amino acids selected from aspartate or glutamate; (d) the amino acid corresponding to position 14 is histidine; (e) the amino acid corresponding to position 17 is histidine; (f) the amino acid corresponding to position 16 is histidine; (g) the amino acid corresponding to position 10 is histidine; (h) the amino acid corresponding to position 10 is asparagine; (i) the amino acid corresponding to position 8 is isoleucine, the amino acid corresponding to position 14 is histidine, and the amino acid corresponding to position 29 is lysine; and preferably the amino acid corresponding to position 46 is cysteine; (j) the amino acid corresponding to position 43 or 46 is cysteine; and/or (k) the amino acid corresponding to position 29 is lysine.
32. The method of paragraph 31, wherein the at least one Ig binding protein comprises a protein having at least 85%, 90%, 95%, or 99% amino acid sequence identity to sequence SEQ ID NO: 1 with at least one of the mutations in (a)-(k) in paragraph 31.
33. The method of any one of paragraphs 21-30, wherein the at least one Ig binding protein comprises a protein having at least 80% an amino acid sequence identity to sequence of SEQ ID NO: 2.
34. The method of any one of paragraphs 21-33, wherein the wash buffer has a pH of about 6.0 to about 10.0 and/or a salt concentration of about 50 mM to about 1000 mM.
35. The method of any one of paragraphs 21-34, wherein the elution liquid has a pH greater than 4.5.
36. A method of viral clearance by affinity chromatography, comprising: providing a liquid sample comprising protein and virus, wherein the protein comprises an Ig sequence; providing an affinity chromatography resin comprising at least one Ig binding protein coupled to thereto; contacting the affinity chromatography resin with the liquid sample under conditions that allow binding of the protein comprising the Ig sequence to the at least one Ig binding protein; washing the affinity chromatography resin with at least one wash buffer, wherein the wash buffer has a pH of about 6.0 to about 10.0 with a salt concentration of about 50 mM to about 1000 mM; and eluting the protein from the affinity chromatography resin with an elution liquid having a pH of about 4 to about 5.5 to produce an eluted protein sample; wherein the eluted protein sample has a reduction in virus compared to the liquid sample.
37. The method of any one of paragraph 36, wherein the at least one Ig binding protein comprises a protein having at least 80% amino acid sequence identity to sequence SEQ ID NO: 1, wherein SEQ ID NO: 1 has at least one of the following mutations: (a) the amino acid corresponding to one or more of position 8 is isoleucine, leucine, valine or an aromatic amino acid; (b) the amino acid corresponding to position 8 is isoleucine or tyrosine; (c) the amino acid corresponding one or more of positions 10, 14, 16, 17, 18, and 28 is/are selected from the group consisting of histidine or acidic amino acids selected from aspartate or glutamate; (d) the amino acid corresponding to position 14 is histidine; (e) the amino acid corresponding to position 17 is histidine; (f) the amino acid corresponding to position 16 is histidine (g) the amino acid corresponding to position 10 is histidine; (h) the amino acid corresponding to position 10 is asparagine; (i) the amino acid corresponding to position 8 is isoleucine, the amino acid corresponding to position 14 is histidine, and the amino acid corresponding to position 29 is lysine; and preferably the amino acid corresponding to position 46 is cysteine; (j) the amino acid corresponding to position 43 or 46 is cysteine; and/or (k) the amino acid corresponding to position 29 is lysine.
38. The method of paragraph 37, wherein the at least one Ig binding protein comprises a protein having at least 85%, 90%, 95%, or 99% amino acid sequence identity to sequence SEQ ID NO: 1 with at least one of the mutations in (a)-(k) in paragraph 37.
39. The method of any one of paragraphs 36-38, wherein the at least one Ig binding protein comprises a protein having at least 80%, preferably at least 95%, of an amino acid sequence identity to sequence of SEQ ID NO: 2.
40. The method of any one of paragraphs 36-38, wherein the at least one Ig binding protein comprises a protein having an amino acid sequence identity to sequence of SEQ ID NO: 2.
41. The method of any one of paragraphs 36-40, wherein the Ig binding protein comprises a cross-linked polysaccharide with a Protein A functional group.
42. The method of any one of paragraphs 36-41, wherein the reduction in virus is at least about 3.8 log₁₀.
43. The method of paragraph 42, wherein the reduction in virus is about 3.8 log₁₀ to about 4.8 log₁₀.
44. The method of any one of paragraphs 36-43, wherein at least a portion of the virus in the first liquid sample remains bound to the affinity chromatography resin when the protein is eluted.
45. The method of any one of paragraphs 36-44, wherein the affinity chromatography resin comprises a cross-linked polysaccharide.
46. The method of paragraph 45, wherein the cross-linked polysaccharide comprises agarose.
47. The method of any one of paragraphs 44-46, wherein the cross-linked polysaccharide is a caustic resistant agarose.
48. The method of any one of paragraphs 36-46, wherein the elution liquid has a pH greater than 4.5.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1: Affinity Chromatography Resins

In this study, three different affinity chromatography resin materials were tested.

### 1. Resin 1

The affinity chromatography resin of Resin 1 is an affinity chromatography resin with uniform agarose beads that are highly cross-linked and modified with a Protein A functional Group. Resin 1 provides elution of one or more mAbs, IgG1, IgG2, IgG4, IgM, IgA, Ig fragments, FC fragments, Fab fragments, fusion proteins comprising an Ig region, and conjugates comprising an Ig region, which can become unstable at pH levels typically used for elution with other Protein A resins (3-3.5). Resin 1 provides alkaline stability (maintains capacity after exposure to 0.1 M to 0.5 M NaOH for 120 hours), capacity of 60 g/L for polyclonal human IgG, and elution up to about pH 5.

In a preferred embodiment, Resin 1 can comprise an Immunoglobulin (Ig) binding protein comprising one or more Ig binding domains corresponding to an Ig binding protein having at least 80%, preferably at least 85%, more preferably at least 90%, even more preferably at least 95%, and most preferably at least 99% amino acid identity to SEQ ID NO: 1, wherein SEQ ID NO: 1 has at least one of the following mutations:
(a) the amino acid corresponding to position 8 of SEQ ID NO: 1 is isoleucine (Ile or I), leucine (Leu or L), valine (V), or an aromatic amino acid;
(b) the amino acid corresponding to position 8 of SEQ ID NO: 1 is isoleucine (Ile or I) or tyrosine (Tyr or Y);
(c) amino acid(s) corresponding to position 10, 14, 16, 17, 18, or 28 of SEQ ID NO: 1 is/are selected from the group of histidine (His or H) or acidic amino acids selected from aspartate (Asp or D) or glutamate (Glu or E); preferably at least one of positions 10, 14, 16, 17, 18, or 28 of SEQ ID NO: 1 is histidine (His or H);
(d) the amino acid corresponding to position 14 of SEQ ID NO: 1 is mutated to histidine (His or H), as shown herein as SEQ ID NO: 5; and preferably position 43 is mutated to cysteine (Cys or C);
(e) the amino acid corresponding to position 17 of SEQ ID NO: 1 is mutated to histidine (His or H), as shown herein as SEQ ID NO: 3; and preferably position 43 is mutated to cysteine (Cys or C);
(f) the amino acid corresponding to position 16 of SEQ ID NO: 1 is mutated to histidine (His or H), as shown herein as SEQ ID NO: 4; and preferably position 43 is mutated to cysteine (Cys or C);
(g) the amino acid corresponding to position 10 of SEQ ID NO: 1 is mutated to histidine, (His or H) as shown herein as SEQ ID NO: 6; and preferably position 43 is mutated to cysteine (Cys or C);
(h) the amino acid corresponding to position 10 of SEQ ID NO: 1 is mutated to asparagine (Asp or D), as shown herein as SEQ ID NO: 7; and preferably position 43 is mutated to cysteine (Cys or C);
(i) the amino acid corresponding to position 8 of SEQ ID NO: 1 is isoleucine (Ile or I), the amino acid corresponding to position 14 of SEQ ID NO: 1 is histidine (His or H), and the amino acid corresponding to position 29 of SEQ ID NO: 1 is lysine (Lys or K), as shown herein as SEQ ID NO: 2; and preferably the amino acid corresponding to position 46 is cysteine (Cys or C;
(j) the amino acid corresponding to position 43 in SEQ ID NO: 1 is cysteine (Cys or C), as shown in each of SEQ ID NO: 3-7; and/or
(k) a cysteine (Cys or C) residue at the position corresponding to position 46 of SEQ ID NO: 1, as shown in SEQ ID NO: 2.

In various embodiments, Resin 1 according to any one of (a)-(k) above can comprise 2, 3, 4, 5, or 6 domains linked to together and/or Resin 1 can be a homomultimer or a heteromultimer.

In a preferred embodiment, an amino acid sequence of Resin 1 is shown below (SEQ ID NO: 1) with at least one of the mutations shown above in paragraphs (a) to (k):

Ile Ala Ala Gln His Asp Lys Asp Gln Gln Ala
Ala Phe Tyr Glu Ile Leu His Leu Pro Asn Leu
Thr Glu Glu Gln Arg Asn Ala Phe Ile Gln Ser
Leu Arg Asp Asp Pro Ser Val Ser Leu Glu Ile
Leu Ala Glu Ala Lys Lys Leu Asn Asp Ala Gln
Ala Pro Lys

In another preferred embodiment, an amino acid sequence of Resin 1 is shown below (SEQ ID NO: 2):

Ala Gln His Asp Lys Ile Gln Gln Ala Ala Phe
His Glu Ile Leu His Leu Pro Asn Leu Thr Glu
Glu Gln Arg Asn Lys Phe Ile Gln Ser Leu Arg
Asp Asp Pro Ser Val Ser Leu Glu Ile Leu Cys
Glu Ala Lys Lys Leu Asn Asp Ala Gln Ala Pro
Lys Ile Ala Ala Gln His Asp Lys Ile Gln Gln
Ala Ala Phe His Glu Ile Leu His Leu Pro Asn
Leu Thr Glu Glu Gln Arg Asn Lys Phe Ile Gln
Ser Leu Arg Asp Asp Pro Ser Val Ser Leu Glu
Ile Leu Ala Glu Ala Lys Lys Leu Asn Asp Ala
Gln Ala Pro Lys

In another preferred embodiment, an amino acid sequence of Resin 1 is shown below (SEQ ID NO: 3):

Ile Ala Ala Gln His Asp Lys Asp Gln Gln Ala
Ala Phe Tyr Glu Ile His His Leu Pro Asn Leu
Thr Glu Glu Gln Arg Asn Ala Phe Ile Gln Ser
Leu Arg Asp Asp Pro Ser Val Ser Leu Cys Ile
Leu Ala Glu Ala Lys Lys Leu Asn Asp Ala Gln
Ala Pro Lys

In another preferred embodiment, an amino acid sequence of Resin 1 is shown below (SEQ ID NO: 4):

Ile Ala Ala Gln His Asp Lys Asp Gln Gln Ala
Ala Phe Tyr Glu His Leu His Leu Pro Asn Leu
Thr Glu Glu Gln Arg Asn Ala Phe Ile Gln Ser
Leu Arg Asp Asp Pro Ser Val Ser Leu Cys Ile
Leu Ala Glu Ala Lys Lys Leu Asn Asp Ala Gln
Ala Pro Lys

In another preferred embodiment, an amino acid sequence of Resin 1 is shown below (SEQ ID NO: 5):

Ile Ala Ala Gln His Asp Lys Asp Gln Gln Ala
Ala Phe His Glu Ile Leu His Leu Pro Asn Leu
Thr Glu Glu Gln Arg Asn Ala Phe Ile Gln Ser
Leu Arg Asp Asp Pro Ser Val Ser Leu Cys Ile
Leu Ala Glu Ala Lys Lys Leu Asn Asp Ala Gln
Ala Pro Lys

In another preferred embodiment, an amino acid sequence of Resin 1 is shown below (SEQ ID NO: 6):

Ile Ala Ala Gln His Asp Lys Asp Gln His Ala
Ala Phe Tyr Glu Ile Leu His Leu Pro Asn Leu
Thr Glu Glu Gln Arg Asn Ala Phe Ile Gln Ser
Leu Arg Asp Asp Pro Ser Val Ser Leu Cys Ile
Leu Ala Glu Ala Lys Lys Leu Asn Asp Ala Gln
Ala Pro Lys

In another preferred embodiment, an amino acid sequence of Resin 1 is shown below (SEQ ID NO: 7):

Ile Ala Ala Gln His Asp Lys Asp Gln Asp Ala
Ala Phe Tyr Glu Ile Leu His Leu Pro Asn Leu
Thr Glu Glu Gln Arg Asn Ala Phe Ile Gln Ser
Leu Arg Asp Asp Pro Ser Val Ser Leu Cys Ile
Leu Ala Glu Ala Lys Lys Leu Asn Asp Ala Gln
Ala Pro Lys

The Ig binding protein according to any one of items (a)-(k) can be immobilized to a solid support. In some embodiments, the Ig binding protein is immobilized to a solid support by a cysteine (C) at the position corresponding to position 43 or 46 of SEQ ID NO: 1.

The Ig binding protein according to any one of items (a)-(k) bind to immunoglobulin with a dissociation constant K_{D} preferably below 200 nM, or below 100 nM, even more preferably 10 nM or less. In some embodiments, the Ig binding protein or Ig binding domain binds to IgG1, IgG2, IgG4, IgM, IgA, Ig fragments, Fe fragments, Fab fragments, fusion proteins comprising an Ig region, and conjugates comprising an Ig region with a dissociation constant K_{D} preferably below 200 nM, or below 100 nM, even more preferably 10 nM or less. Methods for determining binding affinities or binding capacities of Ig binding proteins or domains, i.e. for determining the dissociation constant K_{D}, are known to a person of ordinary skill in the art and can be selected for instance from the following methods known in the art: Surface Plasmon Resonance (SPR) based technology, kinetic exclusion analysis (KinExA assay), Bio-layer interferometry (BLI), enzyme-linked immunosorbent assay (ELISA), flow cytometry, isothermal titration calorimetry (ITC), analytical ultracentrifugation, radioimmunoassay (RIA or IRMA) and enhanced chemiluminescence (ECL). Some of the methods are described further in the Examples. Typically, the dissociation constant K_{D} is determined at 20° C., 25° C., or 30° C. If not specifically indicated otherwise, the K_{D} values recited herein are determined at 22° C.+/−3° C. by surface plasmon resonance spectroscopy. In one embodiment, the Ig binding protein has a dissociation constant K_{D} to human IgG1 in the range between 0.1 nM and 100 nM, preferably between 0.1 nM and 50 nM.

Affinity separation matrix. In another embodiment, the affinity separation matrix can comprise an Ig binding protein or Ig binding domain of the previous embodiments. In preferred embodiments, the affinity separation matrix is a solid support. The affinity separation matrix comprises at least one Ig binding protein or Ig binding domain as described above. An affinity matrix is useful for separation of immunoglobulins and should retain the Ig binding property even after highly alkaline conditions as applied during cleaning processes. Such cleaning of matrices is essential for long-term repeated use of matrices.

Solid support matrices for affinity chromatography are known in the art and include for example but are not limited to, agarose and stabilized derivatives of agarose (e.g., Mabselect®, PrismA®, Sepharose 6B, CaptivA®, rPROTEIN A Sepharose Fast Flow, and others), cellulose or derivatives of cellulose, controlled pore glass (e.g. ProSep® vA resin), monolith (e.g. CIM® monoliths), silica, zirconium oxide (e.g. CM Zirconia or CPG®), titanium oxide, or synthetic polymers (e.g. polystyrene such as Poros 50A or Poros MabCapture® A resin, polyvinylether, polyvinyl alcohol, monodisperse polyacrylate resin (e.g. UniMab™ UniMab™Pro), polyhydroxyalkyl acrylates, polyhydroxyalkyl methacrylates, polyacrylamides, polymethacrylamides etc) and hydrogels of various compositions. In certain embodiments the support comprises a polyhydroxy polymer, such as a polysaccharide. Examples of polysaccharides suitable for supports include but are not limited to agar, agarose, dextran, starch, cellulose, pullulan, etc, and stabilized variants of these.

The formats for solid support matrices can be of any suitable well-known kind. Such solid support matrix for coupling the Ig binding protein or Ig binding domain as described herein might comprise for example, one of the following: columns, capillaries, particles, membranes, filters, monoliths, fibers, pads, gels, slides, plates, cassettes, or any other format commonly used in chromatography and known to someone skilled in the art.

In one embodiment, the matrix is comprised of substantially spherical particles, also known as beads, for example Sepharose or Agarose beads or monodisperse polyacrylate beads. Suitable particle sizes may be in the diameter range of 5-500 μm, such as 10-100 μm, such as 20-80 μm, such as 40-70 μm. Matrices in particle form can be used as a packed bed or in a suspended form including expanded beds.

In an alternative embodiment, the solid support matrix is a membrane, for example a hydrogel membrane. In some embodiments, the affinity purification involves a membrane as matrix to which the Ig binding protein or lg binding domain of the one embodiment is covalently bound. The solid support can also be in the form of a membrane in a cartridge.

In some embodiments, the affinity purification involves a chromatography column containing a solid support matrix to which the Ig binding protein or Ig binding domain of the one embodiment is covalently bound.

Immobilization to a solid support. In embodiments of the invention, the Ig binding protein or Ig binding domain is conjugated to a solid support. In some embodiments, the Ig binding protein or Ig binding domain may comprise additional amino acid residues at the N- and/or C-terminal end. The Ig binding protein or Ig binding domain may be attached to a suitable solid support matrix via conventional coupling techniques. Methods for immobilization of protein ligands to solid supports are well-known in this field and readily performed by the skilled person in this field using standard techniques and equipment. In some embodiments, the coupling may be a multipoint coupling, for example via several lysines, or a single point coupling, for example via cysteine.

In some embodiments, the alkaline stable Ig binding protein or Ig binding domain comprises an attachment site for covalent attachment to a solid phase (matrix). Site-specific attachment sites comprise natural amino acids, such as cysteine or lysine, which enable specific chemical reactions with a reactive group of the solid phase or a linker between the solid phase and the protein.

In some embodiments, the attachment site may be directly at the C- or N-terminal end of the Ig binding protein or Ig binding domain. In some embodiments, a single cysteine is located at the C-terminal end for site-specific immobilization of the Ig binding protein or Ig binding domain. An advantage of having a C-terminal cysteine is that coupling of the Ig binding protein or Ig binding domain can be achieved through reaction of the cysteine thiol with an electrophilic group on a support resulting in a thioether bridge coupling. This provides excellent mobility of the coupled protein which provides increased binding capacity.

In other embodiments, the attachment site may be located in the third helix of the Ig binding protein or Ig binding domain, for example, in position corresponding to position 43 or position 46 of SEQ ID NO: 1.

In other embodiments, there may be a linker between the N- or C-terminus and the attachment site. In some embodiments of the invention, the Ig binding protein or Ig binding domain may comprise a N- or C-terminal amino acid sequence of 3-20 amino acids, preferably of 4-10 amino acids, with a terminal cysteine. Amino acids for a terminal attachment site may be selected from the group of proline, glycine, alanine, and serine, with a single cysteine at the C-terminal end for coupling.

In some embodiments, the Ig binding protein or Ig binding domain may also comprise additional amino acid residues at the N- and/or C-terminal end, such as for example a leader sequence at the N-terminal end and/or a coupling sequence with or without a tag at the N- or C-terminal end.

Use of the Ig binding protein. In one embodiment, the present invention is directed to the use of the Ig binding protein or Ig binding domain of the one embodiment or an affinity matrix of the one embodiment for affinity purification of immunoglobulins or variants thereof, i.e. the Ig binding protein or Ig binding domain is used for affinity chromatography. In some embodiments, the Ig binding protein or Ig binding domain of the invention is immobilized onto a solid support as described in the one embodiment of the invention.

Method of affinity purification of immunoglobulins. Methods of affinity purification of immunoglobulins comprise the following steps:
(a) providing a liquid (sample) that contains an lg such as IgG1, IgG2, IgG4, IgM, IgA, Ig fragments, Fe fragments, or Fab fragments (including fusion proteins and conjugates, as defined above);
(b) providing an affinity separation matrix comprising an immobilized Ig binding protein or Ig binding domain as described above immobilized to said affinity separation matrix;
(c) contacting said liquid with said affinity separation matrix, under conditions that permit binding of the at least one lg binding protein or Ig binding domain as described above to an Ig; and
(d) eluting said Ig from said matrix, thereby obtaining an eluate containing said Ig.

In some embodiments, the method of affinity purification may further comprise one or more washing steps carried out between steps (c) and (d) under conditions sufficient to remove from the affinity separation matrix some or all molecules that are non-specifically bound thereto. Non-specifically bound means any binding that does not involve an interaction between the at least one lg binding protein or Ig binding domain and an Ig.

Affinity separation matrices suitable for the disclosed uses and methods are those matrices according to the embodiments described above and as known to someone skilled in the art. In some embodiments, the elution of the immunoglobulin from (the matrix comprising) the Ig binding protein or Ig binding domain in step (d) is affected through a change in pH and/or a change in salt concentration. In general, suitable conditions for performing the method of affinity purification are well known to someone skilled in the art. In some embodiments, the disclosed uses or methods of affinity purification comprising the disclosed Ig binding proteins or Ig binding domains may provide elution of at least about 95%, at least about 96%, at least about 97%, at least about 98%, at least about 99%, or 100% of Ig containing proteins at a pH of greater than or equal to 3.7 (e.g., about pH 4.0, about pH 4.5, about pH 5.0, or about pH 5.5). Due to the high stability of the Ig binding proteins and Ig binding domains of the invention, solutions with greater than or equal to pH 3.7 can be used for the elution of Ig proteins.

In some embodiments, in step (d) of the method of affinity purification more than 95% of the protein comprising the Ig sequence (e.g. antibody) is eluted at pH 3. 7 or higher (up to about pH 5.5) from the matrix comprising the immobilized Ig binding protein or Ig binding domain as described above. In some embodiments, a further step (e) for efficient cleaning of the affinity matrix is added, preferably by using an alkaline liquid, for example, with pH of 13-14. In certain embodiments, the cleaning liquid comprises 0.1-1.0 M NaOH or KOH, preferably 0.25-0.5 M NaOH or KOH. Due to the high alkaline stability of the lg binding proteins or lg binding domains of the invention, such strong alkaline solution can be used for cleaning purposes. After cleaning the affinity purification matrix with an alkaline cleaning liquid, in some embodiments, at least 88% of the lg binding protein or lg binding domain have Ig binding activity if incubated for at least 48 h at 0.5 M NaOH. In some embodiments, the Ig-binding capacity of the Ig binding protein or Ig binding domain is at least about 80%, at least about 90%, or 100% of the lg binding capacity before the incubation under alkaline conditions, for example, as determined by the remaining Ig-binding capacity after at least 20 h incubation in 0.5 M NaOH.

Methods of isolating an immunoglobulin, comprising the steps (a) contacting a liquid sample comprising an immunoglobulin with a separation matrix comprising a plurality of Ig binding proteins or Ig binding domains (coupled to a solid support); b) washing the separation matrix with a washing liquid at a pH above pH 3.7 (up to 5.5); (c) eluting the immunoglobulin from the separation matrix; and (d) obtaining the immunoglobulin.

Method for the production of an Ig binding protein. In one embodiment the present invention is directed to a method for the production of an Ig binding protein or Ig binding domain of the invention, comprising the step(s): (a) culturing the host cell of the one embodiment under suitable conditions for the expression of the binding protein or Ig binding domain in order to obtain said Ig binding protein or Ig binding domain; and (b) optionally isolating said Ig binding protein or Ig binding domain. Suitable conditions for culturing a prokaryotic or eukaryotic host are well-known to the person skilled in the art.

Ig binding molecules may be prepared by any of the many conventional and well-known techniques such as plain organic synthetic strategies, solid phase-assisted synthesis techniques or by commercially available automated synthesizers. On the other hand, they may also be prepared by conventional recombinant techniques alone or in combination with conventional synthetic techniques.

In some embodiments, the production of the Ig binding protein or Ig binding domain can be performed by cell-free in vitro transcription/translation.

### Expression of Ig Binding Proteins.

BL21 (DE3) competent cells were transformed with an expression plasmid encoding Ig binding proteins. Cells were spread onto selective agar plates (Kanamycin) and incubated either for 2 days at 21° C. or overnight at 37° C. Precultures were inoculated from single colony in 50 ml 2×YT medium supplemented with 50 μg/ml kanamycin and cultured for 17 hours at 37° C. at 210 rpm in a conventional orbital shaker in 250 ml Erlenmeyer flasks. The OD500 readout should be in the range of 3.5-6. Main cultures were inoculated from previous overnight culture with an adjusted start-OD500 of 0.3 in 300 ml superrich medium (modified H15 medium consisting of 2% glucose, 5% yeast extract, 0.89% glycerol, 0.76% lactose, 250 mM MOPS, 202 mM TRIS, 10 mM MgSO4, pH 7.4, antifoam SE15) that was supplemented with 50 μg/ml Kanamycin and trace elements (see Studier 2005) in 1 L thick-walled Erlenmeyer flasks. Cultures were transferred to a resonant acoustic mixer (RAMbio) and incubated at 37° C. with 20×g. Aeration was facilitated by Oxy-Pump stoppers. Recombinant protein expression was induced by metabolizing glucose and subsequently allowing lactose to enter the cells. Cells were grown overnight for approx. 18 hours to reach a final OD500 of about 35-55. Before the harvest, the OD500 was measured, samples adjusted to 0.6/OD500 were withdrawn, pelleted and frozen at −20 C. To collect biomass cells were centrifuged at 12000×g for 20 min at 20° C. Pellets were weighed (wet weight). Cells were stored at −20° C. before processing.

### Purification of Ig Binding Proteins

Ig binding proteins were expressed in the soluble fraction of E. coli. The cells were resuspended in cell disruption buffer and lysed by an ultrasonic cell disruption system (Sonopuls HD 2200, Sandelin). Purification step was performed with IEC Sepharose SP-HP (GE Healthcare) using an AKTAvant system (Ge Healthcare) according to the manufacturer's instructions using citric acid buffer at pH 3.0 (20 mM Citric acid, 1 mM EDTA, pH 3.0). Pure protein fractions were eluted by increasing sodium chloride concentration to 1 M with a linear gradient in 10 column volumes. Furth purification was performed by size exclusion chromatography (Superdex 75) according to manufactures instructions using citric acid buffer pH 6.0 (20 mM Citric acid, 150 mM NaCl, 1 mM EDTA, pH 6.0). Results: The purity of variants 81 and 6L was >95% after SE-HPLC and >90% after RP HPLC.

### SEQ ID NO: 2 as Ligand for Affinity Purification of IgG

Affinity for hIgG1: For the analysis of affinity ligand SEQ ID NO: 2, the monoclonal antibodies Cetuximab (IgG1) and Belimumab (IgG1) were used as target. The K_{D} of SEQ ID NO: 2 for IgG Cetuximab was 40.8 nM and for IgG1 Belimumab 47.4 nM.

Binding capacity: Binding capacity was determined with IgG1 sample. The antibody was injected onto coupled resin with affinity ligand SEQ ID NO: 2 until 10% target breakthrough at 6 min residence time. Loaded antibody was quantified and calculated as dynamic binding capacity DBC10%, The DBC10% at 6 min residence time with 2.2 mg/ml Belimumab compared to cs26 was 104.1% compared to cs26.

Caustic stability: Resin 1 with immobilized 19.6 mg/ml of affinity ligand of SEQ ID NO: 2 (coupled at pH=10.5 and 2.05 M Na2SO4) was incubated with 0.5 M NaOH for 24 h at room temperature (22° C.+/−3° C.). Even after 24 h in strong alkaline solution, SEQ ID NO: 2 showed no reduction in binding capacity for Ig (99%).

Elution pH: Elution pH of peak maximum was determined as described above. SEQ ID NO: 2 showed high elution pH (pH 5.0) for Gammanorm and Belimumab in comparison to cs26 (pH 3.5), see Table 4.

TABLE 4
Elution of peak maximum.

| | Elution pH | Elution pH |
|---|---|---|
| | peak maximum; | peak maximum; |
| Affinity Ligand | Gammanorn | Belimumab |
| SEQ ID NO: 2 | 5.0 | 5.0 |
| cs26 | 3.5 | 3.5 |

Step elution was analyzed for target elution at pH 4.8. Residual elution of the target (Gammanorm or Belimumab) from the ligand (SEQ ID NO: 2) was analyzed after 100 mM phosphoric acid CIP at pH 1.7 (recovery). The target protein was nearly completely eluted from the ligand of SEQ ID NO: 2 at pH 4.8, see Table 5.

TABLE 5
Target elution at pH 4.8 compared to pH 1.7

| | Elution Recovery | Elution Recovery |
|---|---|---|
| | pH 4.8 vs. 1.7 | pH 4.8 vs. 1.7 |
| Affinity Ligand | Gammanorm | Belimumab |
| SEQ ID NO: 2 | 96.9% | 99.7% |
| cs26 | 15.8% | 19.3% |

### 2. Resin 2, a Comparative Example to Resin 1

Resin 2 is an affinity chromatography resin comprising a highly cross-linked, alkaline stable, uniform agarose base matrix with a modified Protein A ligand. Resin 2 can be used for protein and monoclonal antibody purification. Resin 2 is used as a comparative example to Resin 1.

### 3. Mab Select SuRe LX Resin

(Cytiva, cytivalifesciences.com/en/us/shop/chromatography/resins/affinity-antibody/mabselect-sure-lx-protein-a-resin-p-00628) is an affinity chromatography resin comprising alkali-tolerant rProtein A ligand that withstands clean-in-place and sanitization procedures with 0.1 to 0.5 M NaOH. Mab Select SuRe LX can be used for purification of monoclonal antibodies for clinical applications.

The chromatography details for each resin are presented in Table 6.

TABLE 6
Chromatography Column Parameters

| Parameter | Small Scale Model |
|---|---|
| Column Diameter (cm) | 1.0 |
| Column Bed Height (cm) | 20 |
| Column Volume | 15.7 mL |
| Linear Flow Rate (cm/h) | 300 for process |
| | 200 for loading |
| Volumetric flow rate | 3.93 mL/min for process |
| | 2.62 mL/min for loading |
| Load concentration (g/L) | 2.8 |
| Load Ratio | 45 g of product per L of resin |

### Example 2: Chromatography Method

Liquid samples used in chromatography runs described herein comprised an IgG4 class antibody that was expressed as a secreted protein in mammalian cell culture using a Chinese Hamster Ovary (CHO) cell line. The liquid samples were a harvested cell culture fluid (HCCF) from the CHO cell line comprising the secreted IgG4 that was obtained from a bioreactor. The liquid samples were dispensed into irradiated polyethylene terephthalate (PETG) bottles and stored frozen (−80° C.±10° C.) before actual use.

The harvested cell culture fluid (HCCF) from the CHO cell line comprising the secreted IgG4 After the chromatography runs, eluates were tested for step percent protein recovery & Size Exclusion High Performance Liquid Chromatography (SE-HPLC). Below Table 7 shows the comparison between small scale run results.

Chromatography runs were carried out in duplicates and the operating conditions are detailed in Table 7.

TABLE 7
Small-scale method

| | | Target | Flow | | |
|---|---|---|---|---|---|
| | | volume | rate | Estimated | Fraction |
| Step | Solution | (CV) | (cm/h) | time (mins) | collection |
| Rinse | Purified water | 2 | 160 | 15 | To waste |
| Pre-Use | 0.5M Sodium | 3 | 240 | 15 | 15 minute |
| Sanitization | Hydroxide | | | | contact time |
| | | | | | (under flow no |
| | | | | | static hold) |
| Rinse | Purified water | 2 | 300 | 8 | To waste |
| Neutralisation | 20 mM Sodium | 2 | 300 | 8 | N/A |
| | phosphate, 150 mM | | | | |
| | sodium chloride, | | | | |
| | pH 7.4 | | | | |
| Equilibration | 20 mM Sodium | 4 | 300 | 15 | Pause at 3.5 |
| | phosphate, 150 mM | | | | CVs for offline |
| | sodium chloride, | | | | pH check to |
| | pH 7.4 | | | | confirm column |
| | | | | | effluent |
| | | | | | within ±0.2 pH |
| | | | | | units of buffer |
| Load | Pro A Load Target | Variable | 200 | Variable | To waste |
| | load ratio: 45 g/L | | | | |
| Post Load | 20 mM Sodium | 1.5 | 200 | 9 | |
| wash 1 | phosphate, 500 mM | 3.5 | 300 | 14 | |
| | sodium chloride, | | | | |
| | pH 7.4 | | | | |
| Post Load | 50 mM Tris, 1M | 5 | 300 | 20 | |
| wash 2 | sodium chloride | | | | |
| | pH 8.0 | | | | |
| Post Load | 50 mM Sodium | 3 | 300 | 12 | |
| Wash 3 | phosphate, pH 6.0 | | | | |
| Elution | 50 mM Sodium | 6 | 300 | 24 | Elution peak |
| | acetate, pH 3.5 Or | | | | collection begins |
| | 50 mM Sodium | | | | when UV reaches |
| | acetate pH 4.5 | | | | 20 mAU. End |
| | | | | | collection when |
| | | | | | UV falls to 20 |
| | | | | | mAU (for a 2 mm |
| | | | | | path length) |
| | | | | | Product sample |
| | | | | | tested for virus |
| Strip | 100 mM Acetic Acid | 2 | 300 | 8 | To waste |
| Rinse | Purified water | 2 | 300 | 12 | |

Performed for all Small-Scale model qualification runs

| | | | | | |
|---|---|---|---|---|---|
| Post use | 0.5M Sodium | 3 | 240 | 15 | To waste |
| sanitization | hydroxide | | | | |
| Regeneration | 20 mM Sodium | 4 | 300 | 16 | |
| | phosphate, 150 mM | | | | |
| | sodium chloride, | | | | |
| | pH 7.4 | | | | |
| Storage | 20% v/v Ethanol | 4 | 160 | 30 | |

After the chromatography runs, eluates were tested for step percent protein recovery & Size Exclusion High Performance Liquid Chromatography (SE-HPLC). Below Table 8 shows the comparison between small scale run results.

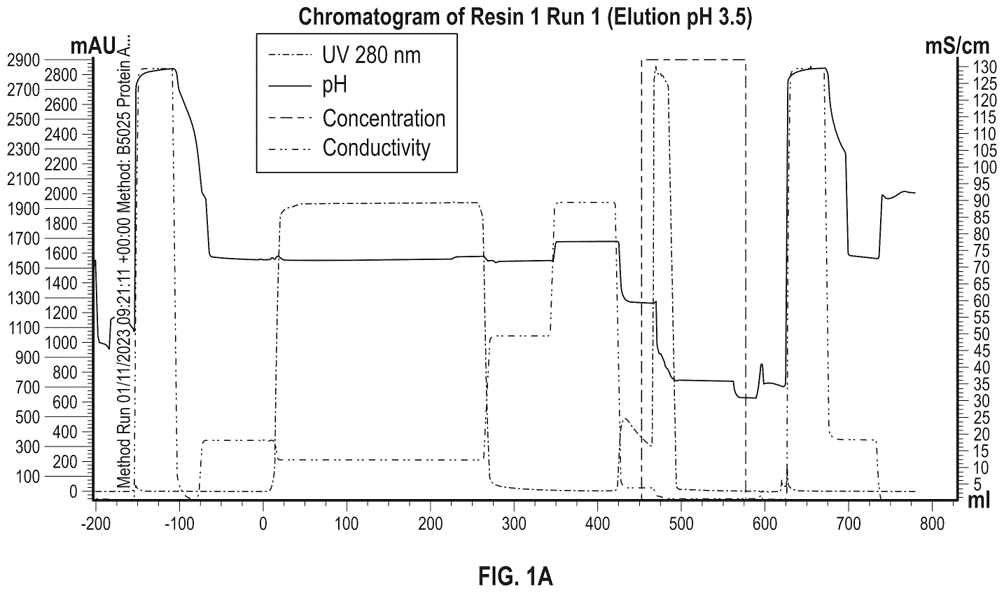

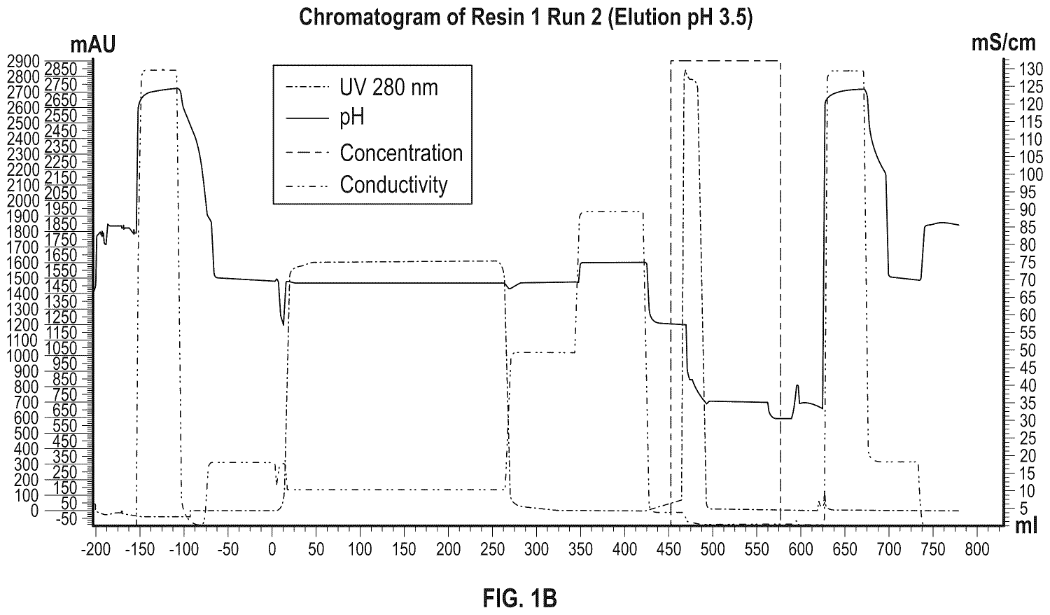

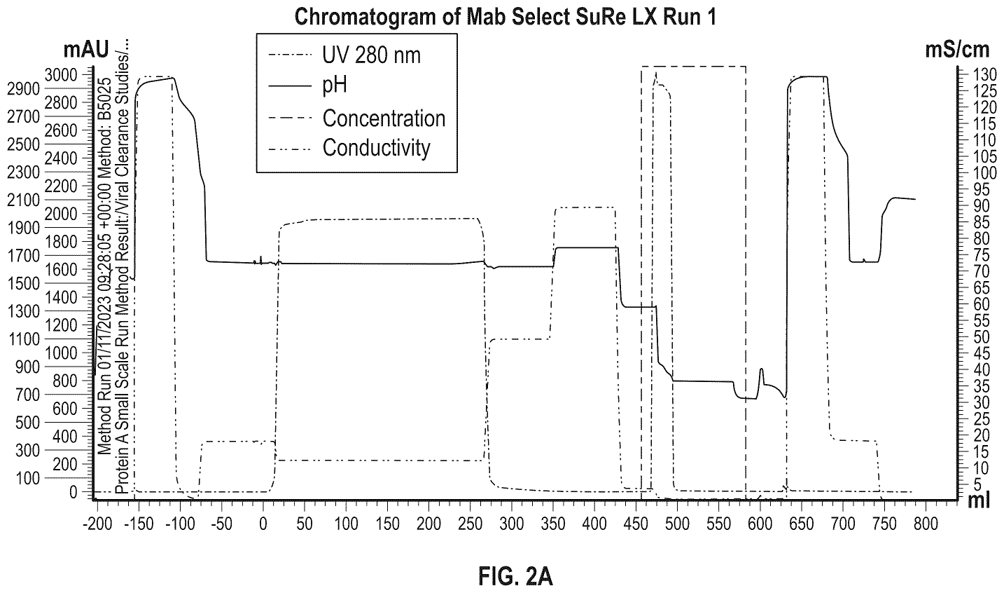

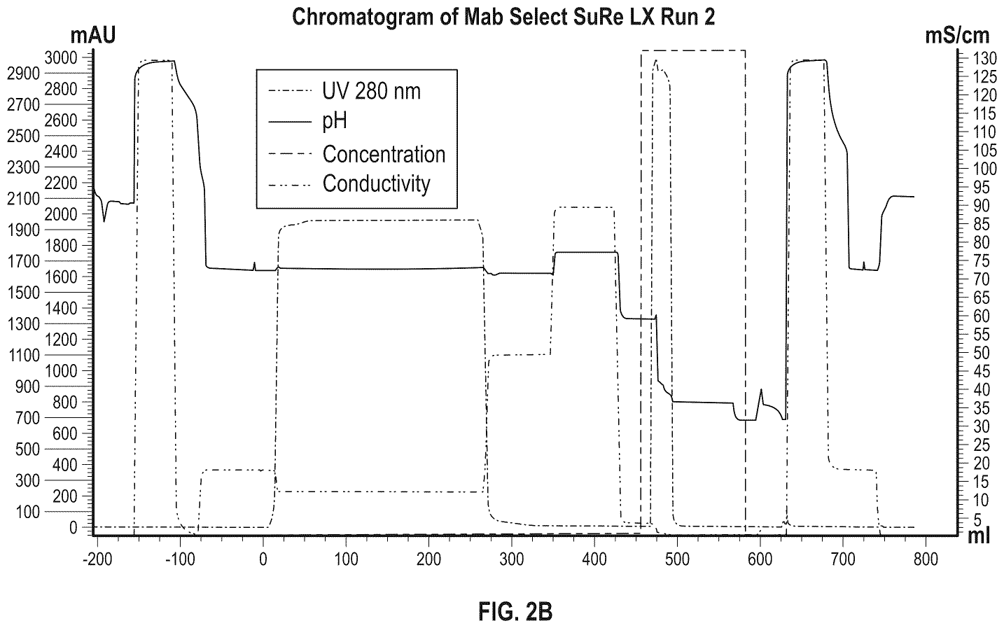

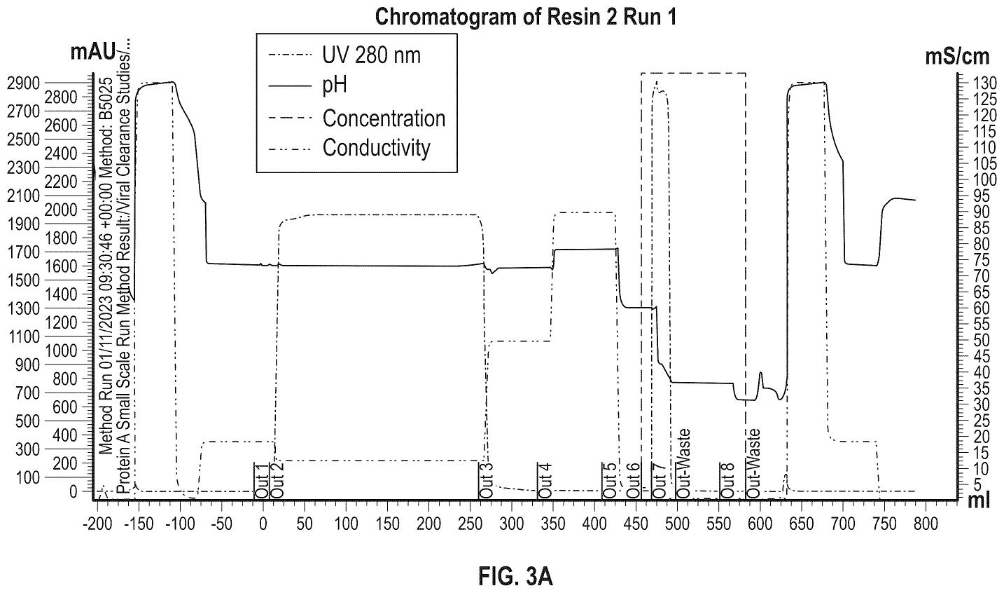

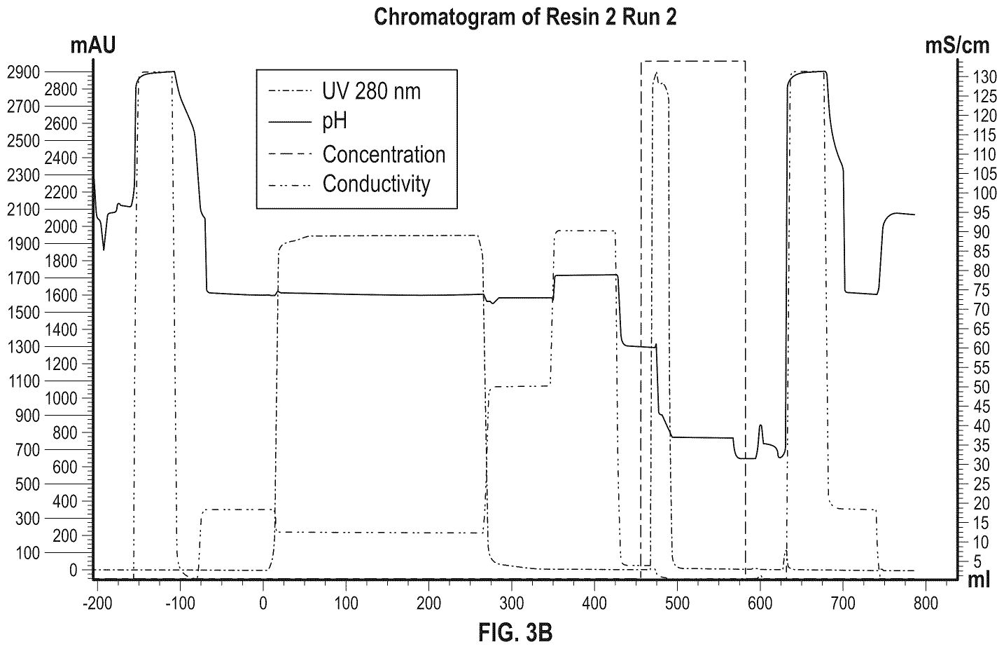

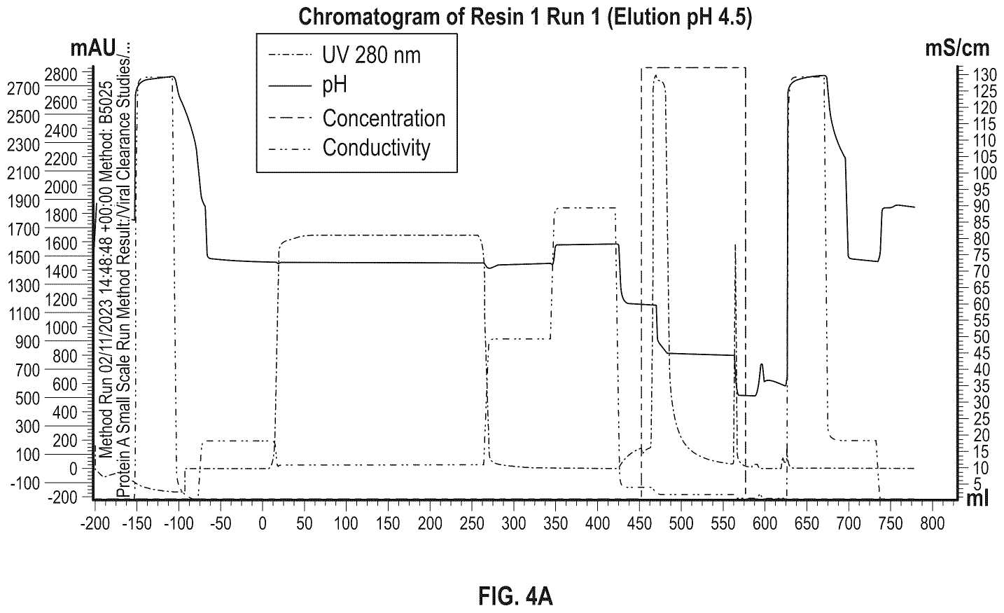

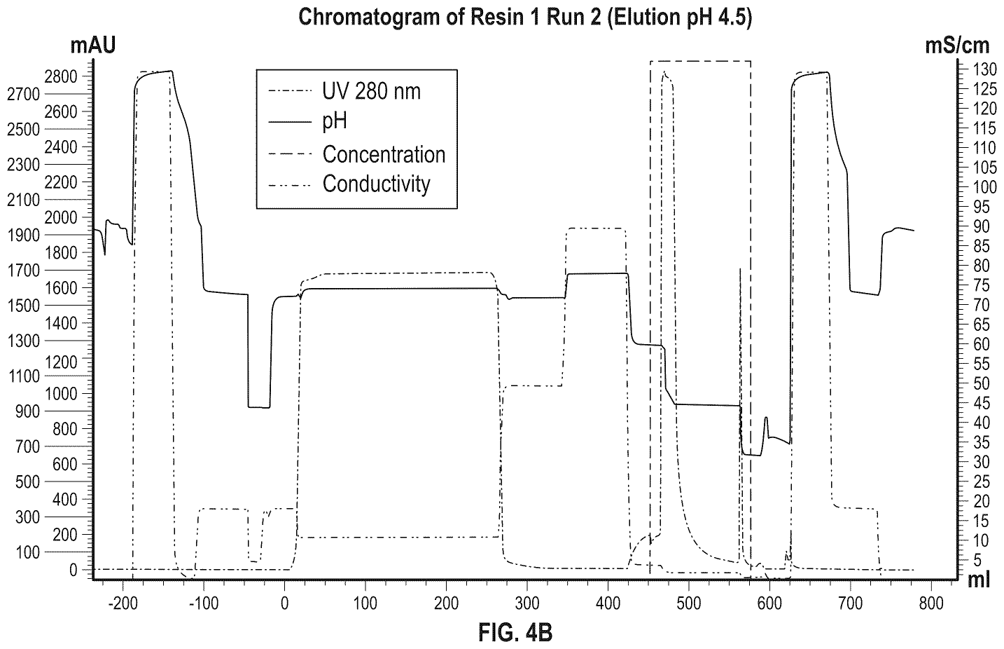

TABLE 8
Summary of small-scale model run results

| | | Step | SE-HPLC | | | |
|---|---|---|---|---|---|---|
| | Batch | Yield | LMW | Main | HMW | Chromatogram |
| Resin | No. | (%) | (%) | Peak (%) | (%) | appearance |
| Resin 1 (pH | Run 1 | 97.12 | 0.615 | 86.995 | 12.391 | FIG. 1A |
| 3.5 elution) | Run 2 | 82.44 | 0.842 | 98.034 | 1.124 | FIG. 1B |
| Mab Select | Run 1 | 105.66 | 0.958 | 98.195 | 0.847 | FIG. 2A |
| SuRe LX | Run 2 | 103.48 | 0.943 | 98.232 | 0.825 | FIG. 2B |
| Resin 2 | Run 1 | 104.69 | 1.000 | 98.214 | 0.786 | FIG. 3A |
| (comparative | Run 2 | 101.58 | 0.905 | 98.335 | 0.760 | FIG. 3B |
| example to | | | | | | |
| Resin 1) | | | | | | |
| Resin 1 (pH | Run 1 | 84.00 | 0.341 | 66.201 | 33.458 | FIG. 4A |
| 4.5 elution) | Run 2 | 83.01 | 0.321 | 65.406 | 34.273 | FIG. 4B |

For Resin 1 runs, it was observed that the bound protein (IgG4 class antibody) leached out during wash 3 buffer (50 mM Sodium phosphate, pH 6.0) which may account for the lower yields of these runs. To avoid this protein loss, the pH of wash 3 buffer was increased from pH 6.0 to pH 7.0 while executing the virus spiking runs, as shown below in Example 3.

### Example 3: Viral Clearance by Protein A Chromatography Resins

Virus. The viruses included in the study are listed in Table 9. The virus panel was selected according to principles of current industry guidelines International Conference on Harmonisation (ICH) of technical requirements for registration of pharmaceuticals for human use, Guideline ICH Q5A (database.ich.org/sites/default/files/Q5A_R1_Guideline.pdf). The aim was to determine log reduction values (LRV) for viral clearance for two model viruses, one enveloped (MLV) and one non-enveloped (MMV).

TABLE 9
Viruses Used for Spike-In for Virus Clearance
Testing of Protein A Chromatography Resins

| | | | | | Size | |
|---|---|---|---|---|---|---|
| Model Virus | Strain | Taxonomy | Genome | Structure | (nm) | Stability |
| Xenotropic Murine | pNFS Th-1 | Retroviridae | ssRNA | Enveloped | 80-110 | Low |
| Leukemia Virus | | | | | | |
| (MLV) | | | | | | |
| Murine Minute | Prototype | Parvoviridae | ssDNA | Non- | 18-24 | Very high |
| Virus (MMV) | | | | enveloped | | |

Virus Clearance Protocol. The performance of an affinity chromatography resin of Resin 1 was compared with two other commercially available example Protein A resins (Resin 2 and Mab SelectSuRe LX from Cytiva) for virus clearance. A summary of the runs performed, and the elution conditions tested, are shown in Table 10.

TABLE 10
Viral Clearance Study Overview

| | | | MMV |
|---|---|---|---|
| | | MLV Enveloped | Non enveloped |
| | | Retrovirus | Parvovirus |
| | | Number of | Number of |
| | Elution | Chromatography | Chromatography |
| Resin | Condition | Runs | Runs |
| Resin 2 | pH 3.5 | 2 | 2 |
| (comparative | | | |
| example to | | | |
| Resin 1) | | | |
| Resin 1 | pH 3.5 | 2 | 2 |
| Resin 1 | pH 4.5 | 2 | 2 |
| MabSelect | pH 3.5 | 2 | 2 |
| SuRe LX | | | |

| | | |
|---|---|---|
| Total runs per virus | 8 | 8 |

Duplicate runs for each virus and condition were performed to confirm reproducibility of the virus reduction factors obtained. This was confirmed by the log reduction factors given from each duplicate run being within 1 log of each other. Duplicate runs were performed in parallel on two separate ÄKTA™ Pure 25 chromatography systems and each run was performed using a new pre-packed column to avoid any viral carry over between runs as cleaning has not been validated for viral removal.

The harvested cell culture fluid (HCCF) from the CHO cell line comprising the secreted IgG4 (i.e., liquid sample) for the spiking runs was taken from 200 L batch (B4123, NBJ2004-23-46) with a protein concentration prior to spike of 2.8 g/L. Bulk aliquots of the liquid sample were thawed, pooled together, filtered and aliquoted within a laminar flow cabinet (LAF cabinet) into volumes that allow for one bottle to be used per run. Prior to use, the required volume of liquid sample was thawed in a water bath set at 18-22° C. and equilibrated to process temperature (20±5° C.). Prior to spiking the liquid sample with virus, the liquid sample was filtered through a 0.22 μm polyethersulfone (PES) membrane.

The small-scale chromatography runs during the virus spiking study were carried out as described in Table 11.

TABLE 11
Small-scale method for viral clearance study.

| | | Target | Flow | | |
|---|---|---|---|---|---|
| | | volume | rate | Estimated | Fraction |
| Step | Solution | (CV) | (cm/h) | time (mins) | collection |
| Rinse | Purified water | 2 | 160 | 15 | To waste |
| Pre-Use | 0.5M Sodium | 3 | 240 | 15 | 15 minute |
| Sanitization | Hydroxide | | | | contact time |
| | | | | | (under flow no |
| | | | | | static hold) |
| Rinse | Purified water | 2 | 300 | 8 | To waste |
| Neutralization | 20 mM Sodium | 2 | 300 | 8 | N/A |
| | phosphate, 150 mM | | | | |
| | sodium chloride, | | | | |
| | pH 7.4 | | | | |
| Equilibration | 20 mM Sodium | 4 | 300 | 15 | Pause at 3.5 |
| | phosphate, 150 mM | | | | CVs for offline |
| | sodium chloride, | | | | pH check to |
| | pH 7.4 | | | | confirm column |
| | | | | | effluent |
| | | | | | within ±0.2 pH |
| | | | | | units of buffer |
| Load | Pro A Load Target | Variable | 200 | Variable | To waste |
| | load ratio: 45 g/L | | | | |
| Post Load | 20 mM Sodium | 1.5 | 200 | 9 | |
| wash 1 | phosphate, 500 | 3.5 | 300 | 14 | |
| | mM sodium | | | | |
| | chloride, pH 7.4 | | | | |
| Post Load | 50 mM Tris, 1M | 5 | 300 | 20 | |
| wash 2 | sodium chloride | | | | |
| | pH 8.0 | | | | |
| Post Load | 50 mM Sodium | 3 | 300 | 12 | |
| Wash 3 | phosphate, pH 7.0 | | | | |
| Elution | 50 mM Sodium | 6 | 300 | 24 | Elution peak |
| | acetate, pH 3.5 Or | | | | collection begins |
| | 50 mM Sodium | | | | when UV reaches |
| | acetate pH 4.5 | | | | 20 mAU. End |
| | | | | | collection when |
| | | | | | UV falls to 20 |
| | | | | | mAU (for a 2 mm |
| | | | | | path length) |
| | | | | | Product sample |
| | | | | | tested for virus |
| Strip | 100 mM Acetic | 2 | 300 | 8 | To waste |
| | Acid | | | | |
| Rinse | Purified water | 2 | 300 | 12 | |

Determining Virus Titer in Eluate. Samples of the spiked liquid sample and eluate (eluted sample) for each run were tested for virus titer by determining the 50% Tissue Culture Infectious Dose (TCID50) infectivity assay or quantitative polymerase chain reaction (QPCR) analysis. Virus log reduction factors were then calculated for each resin & condition tested.

To determine the virus reduction factor, an assay that does not discriminate between infectious and inactivated virus was used. Therefore, QPCR analysis has been used to determine MLV titers for the Protein A Chromatography samples, as the eluted sample was eluted at low pH which may have an inactivating effect or partially inactivating effect on enveloped virus. This approach allows for the reduction factor from the Protein A step to be included in process reduction factors where there is a low pH viral inactivation step included.

For the virus spiked runs, one small scale pre-packed column containing new unused resin was used for each spiked run, and duplicate runs were performed in parallel using two AKTA Pure 25 chromatography systems. AKTA systems were flushed and decontaminated between runs using 1 M NaOH and a static hold of at least 1 hour. The spiked chromatography runs were performed according to AJ18RE-B (Table 9) with a change of wash 3 buffer pH to 7.0 and details are recorded in the ELN. During performance of the spike chromatography runs using Resin 1 there was an increase in the UV A280 response during Post load wash 3 similar to that seen in the small-scale model runs detailed in section 4. The UV response rose to about 100 mAU and plateaued at this level for the remaining volume of the wash. As this was above the collection trigger for the elution peak an autozero of the UV detector was executed at the end of Wash 2 to bring the UV level down to below the collection trigger. The aim of this was to keep the start of collection of the product peak comparable between the runs performed with all the resins. The total virus loaded onto each small-scale column is shown below in Table 12.

TABLE 12
Total Virus loaded over Protein A Small-scale model runs.

| Resin | MMV (by TCID₅₀) | MLV (by QPCR) |
|---|---|---|
| Resin 2 | 8.34 ± 0.36 log₁₀ | 11.35 ± 0.04 log₁₀ |
| Resin 1 (Elution pH 3.5) | 8.43 ± 0.33 log₁₀ | 11.40 ± 0.04 log₁₀ |
| Resin 1 (Elution pH 3.5) | 8.25 ± 0.38 log₁₀ | 11.39 ± 0.04 log₁₀ |
| Mab Select SuRe LX | 8.52 ± 0.28 log₁₀ | 11.23 ± 0.04 log₁₀ |

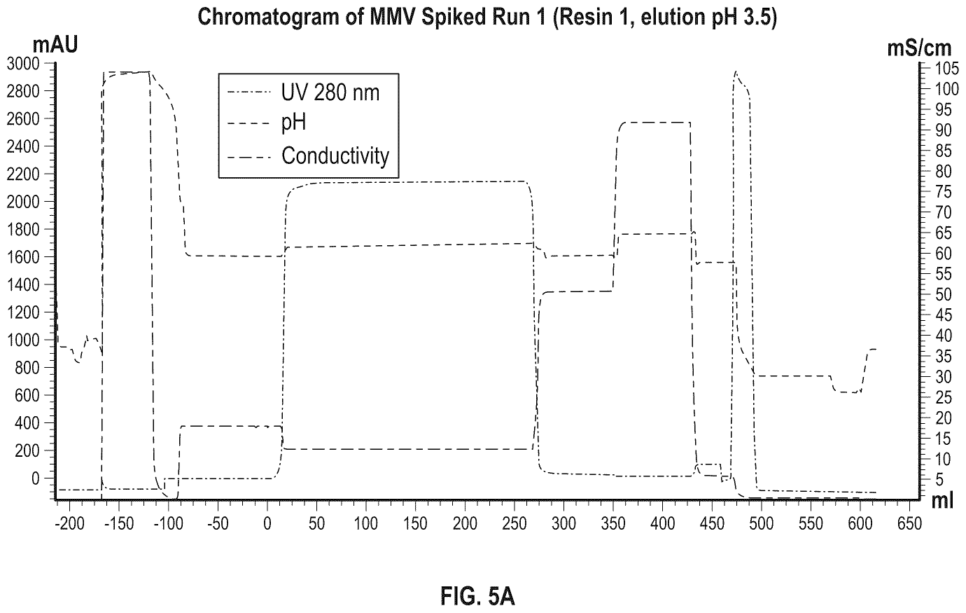

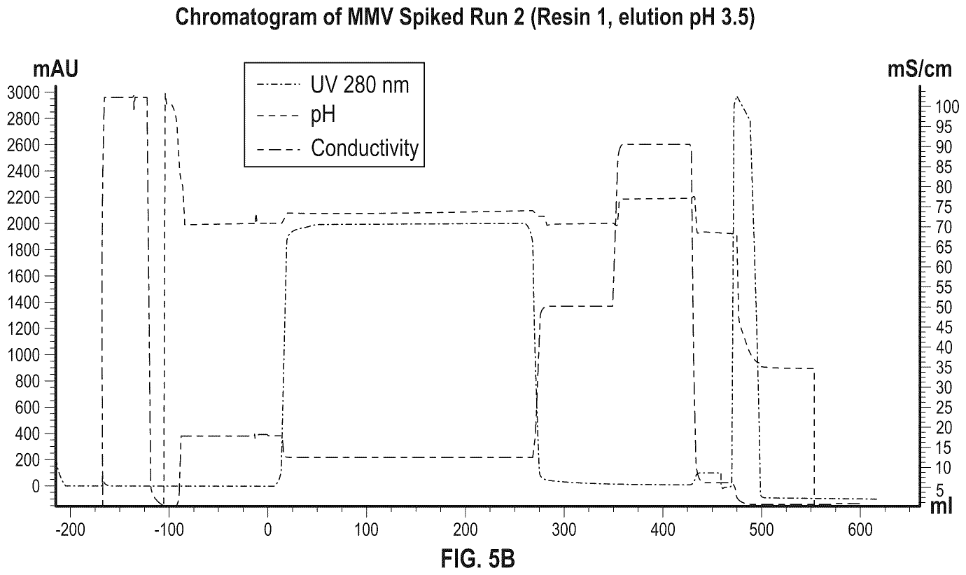

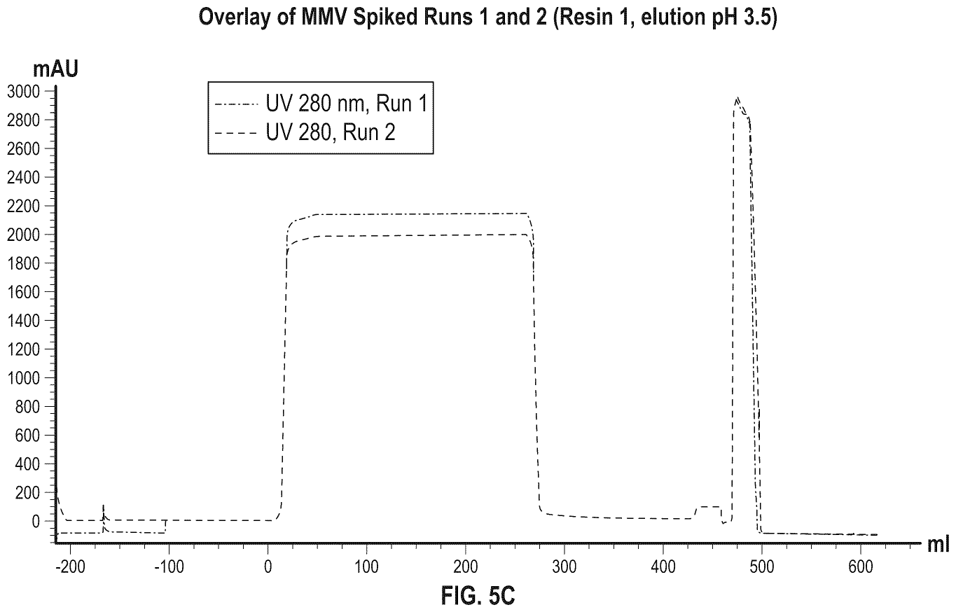

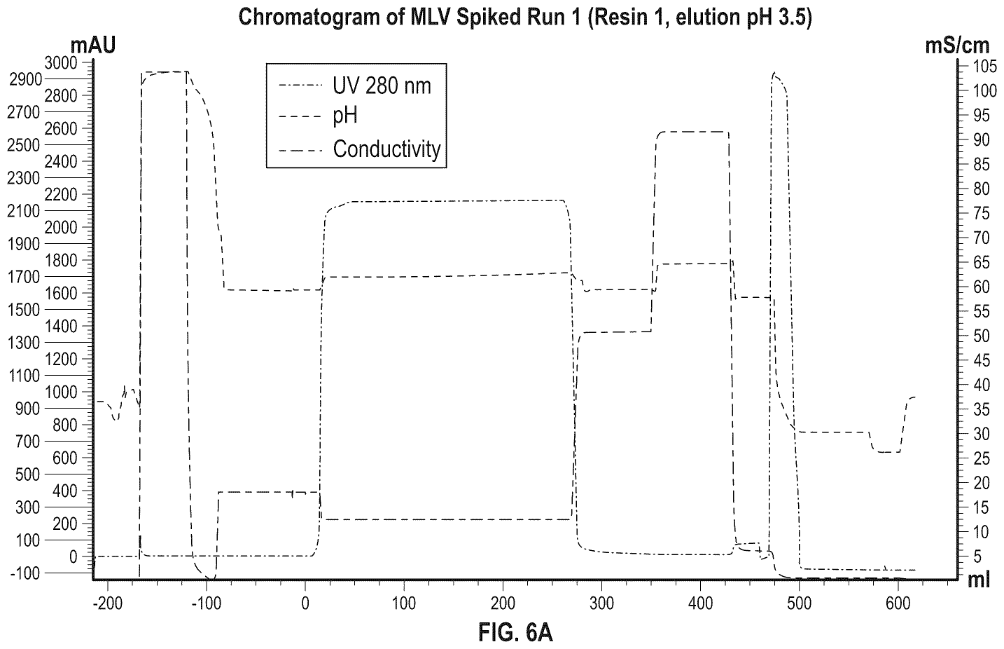

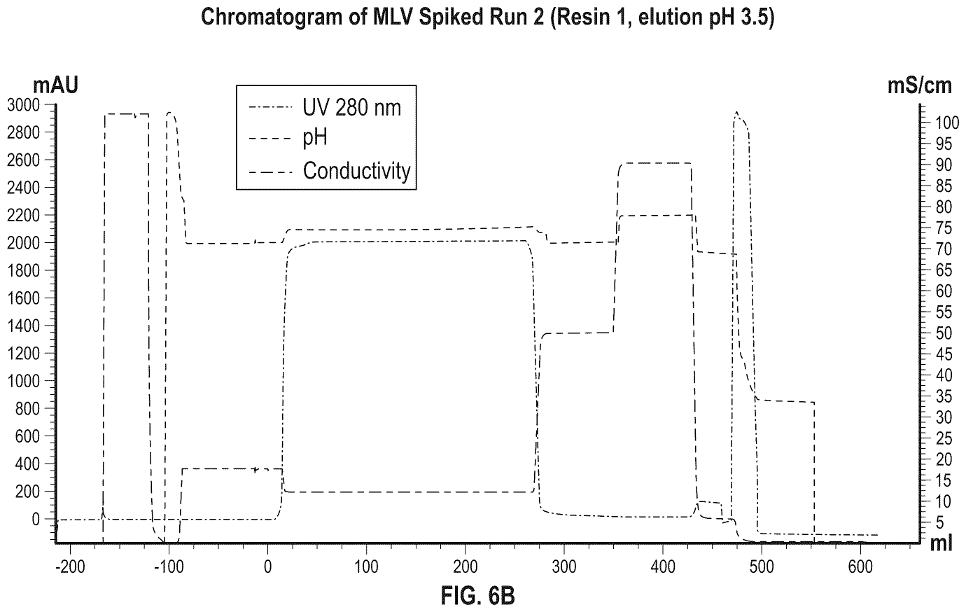

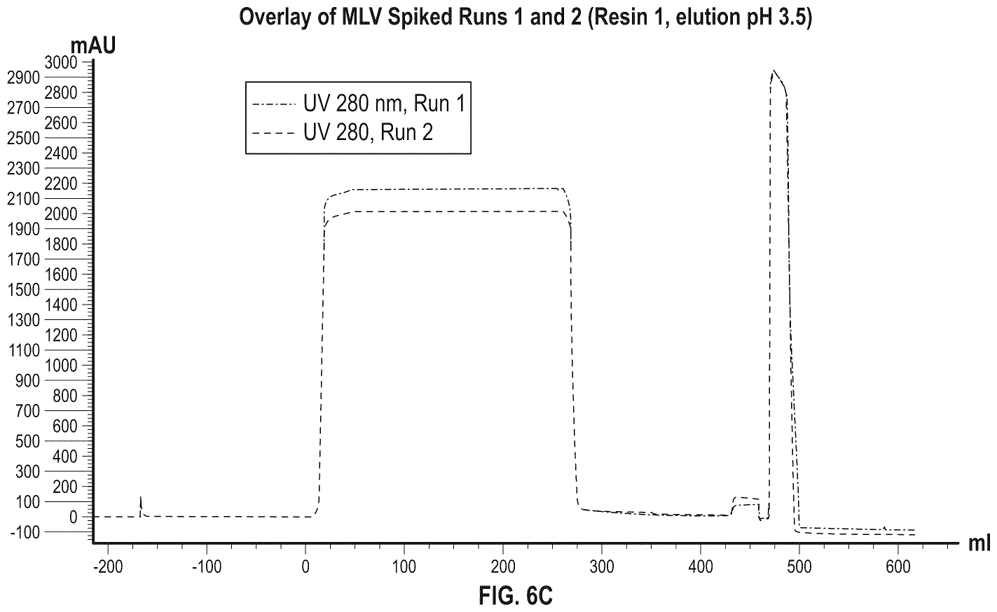

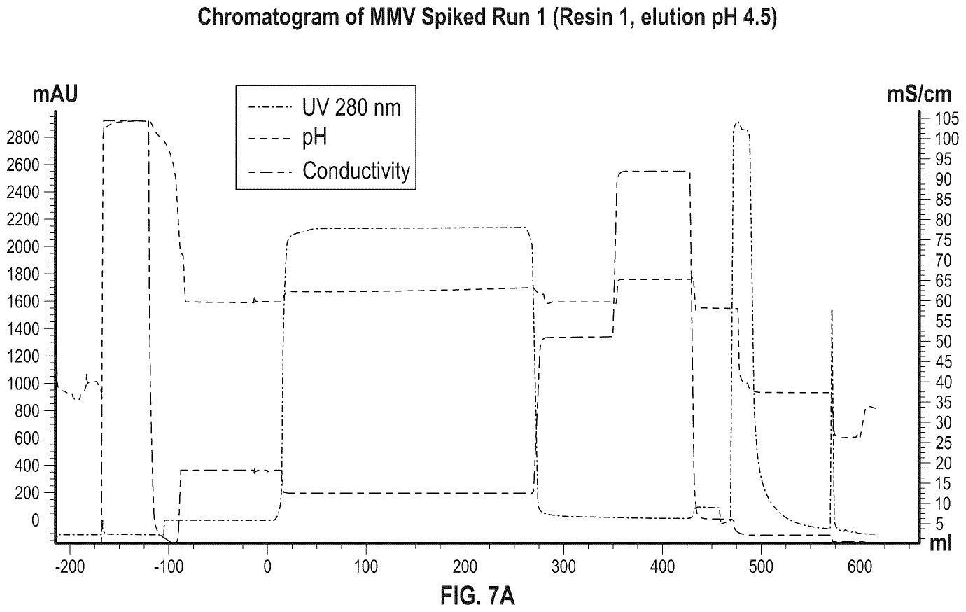

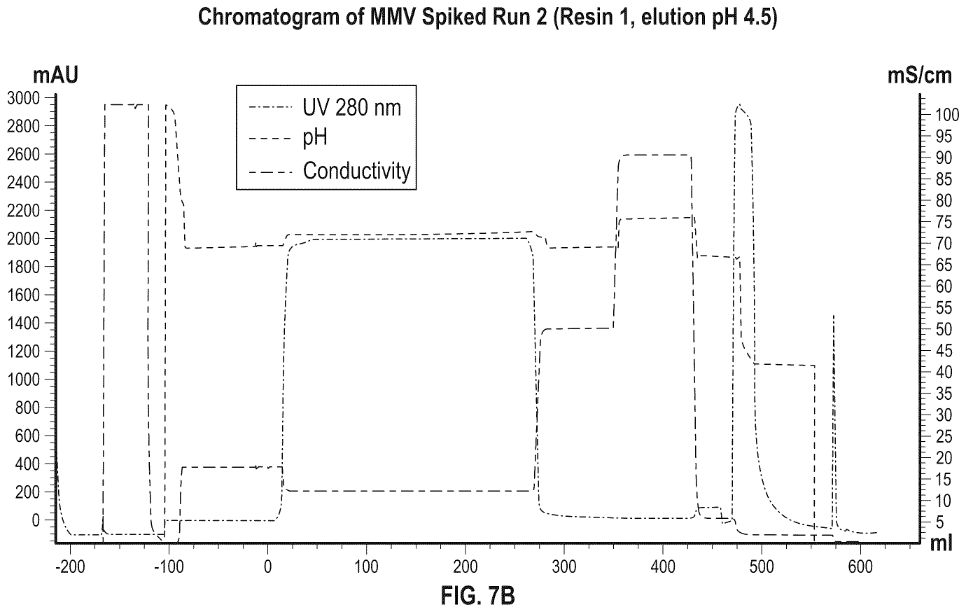

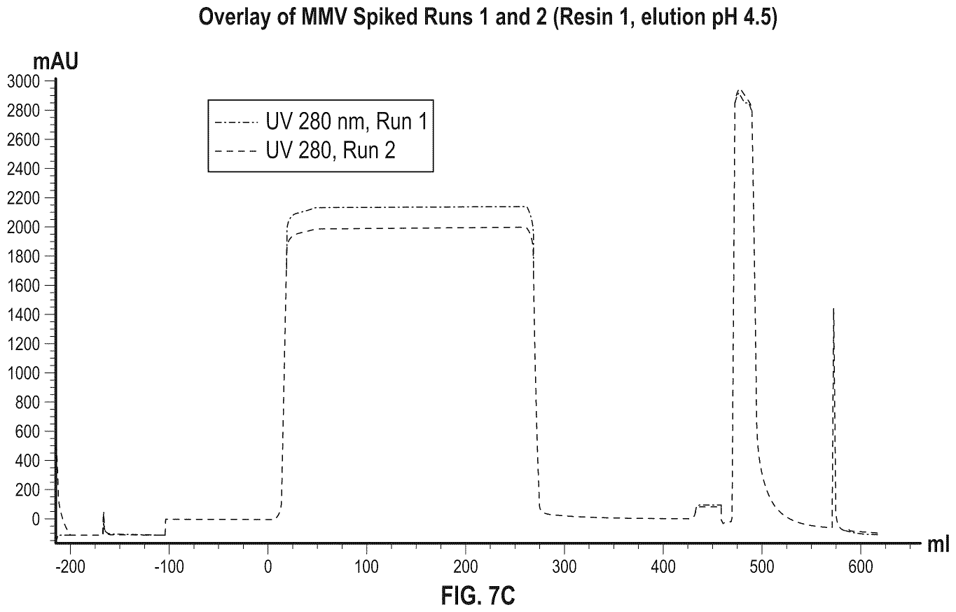

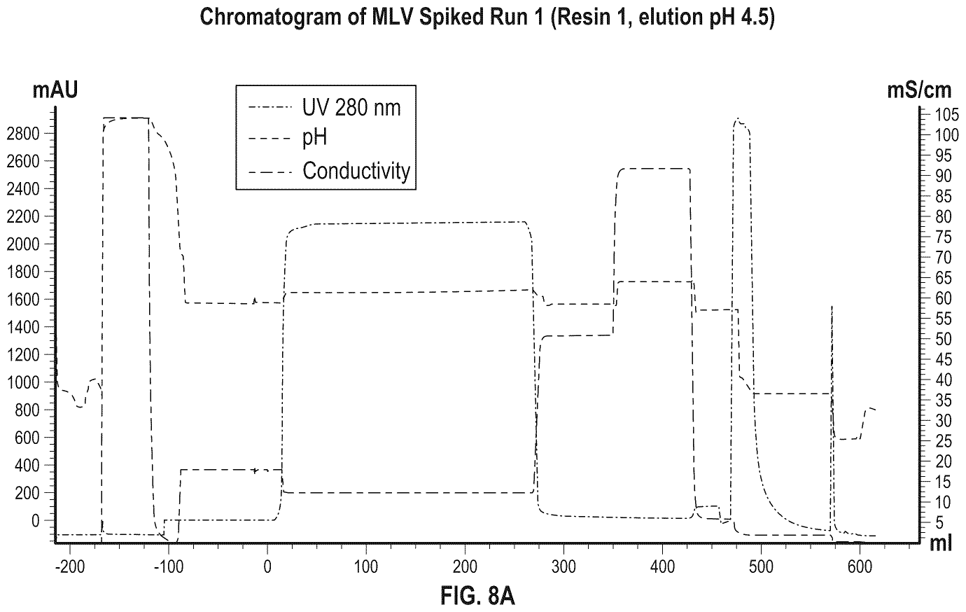

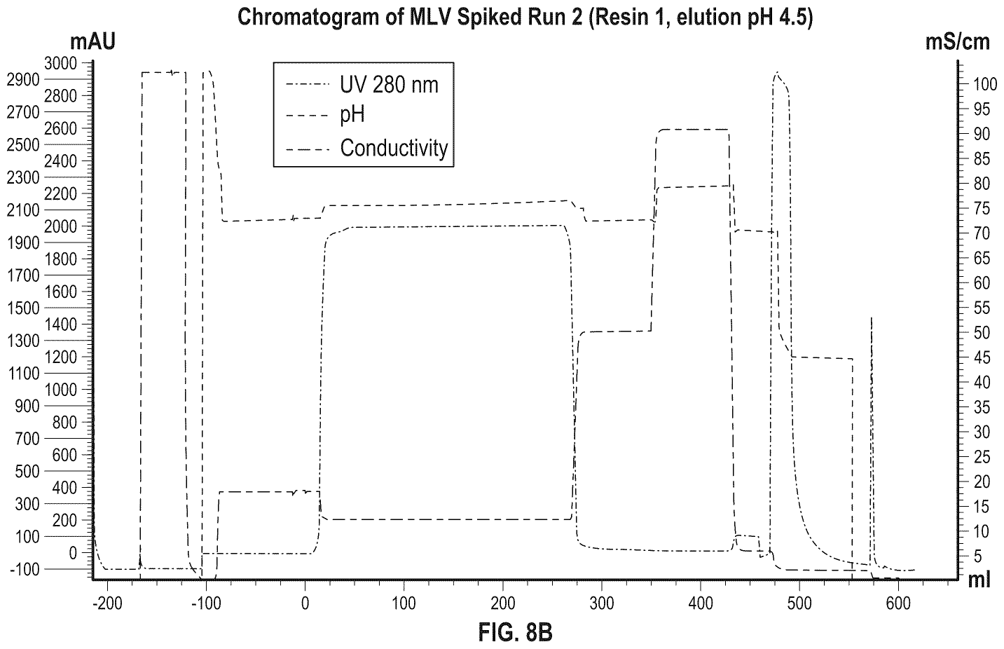

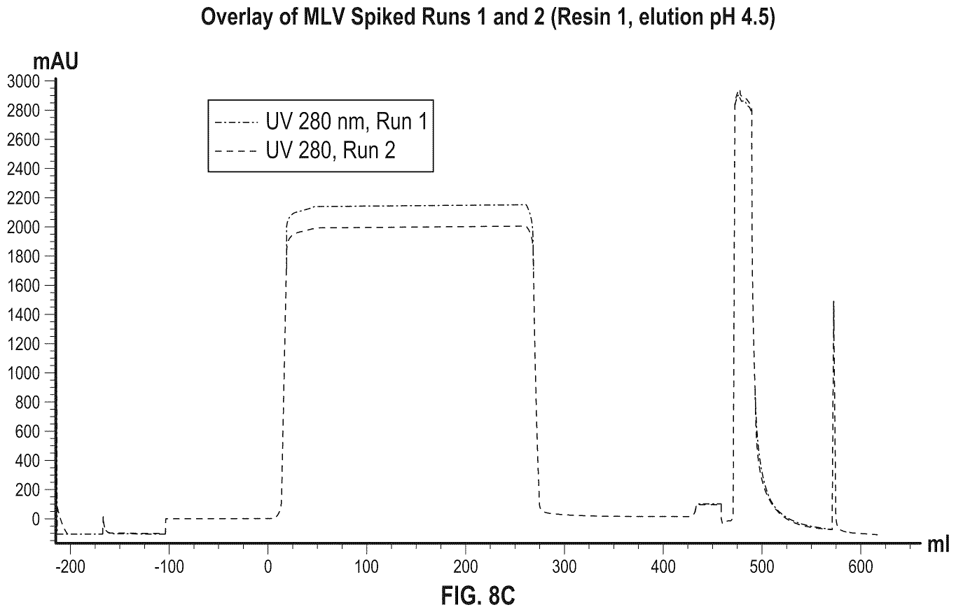

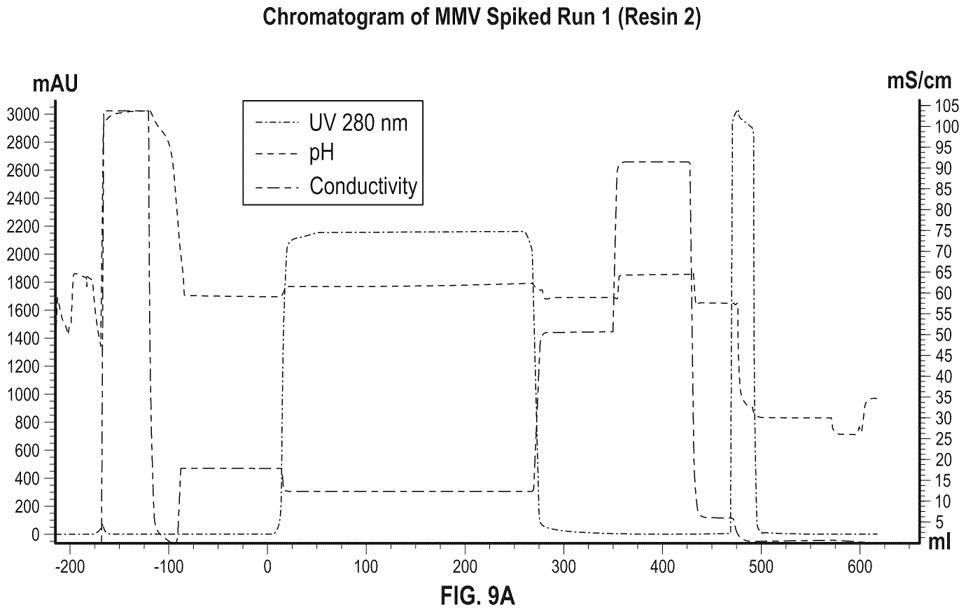

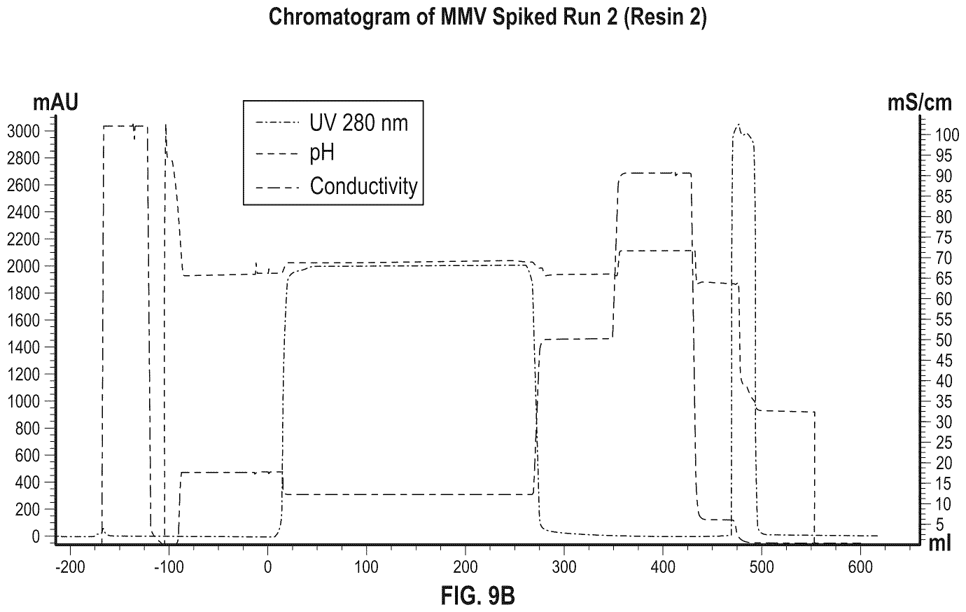

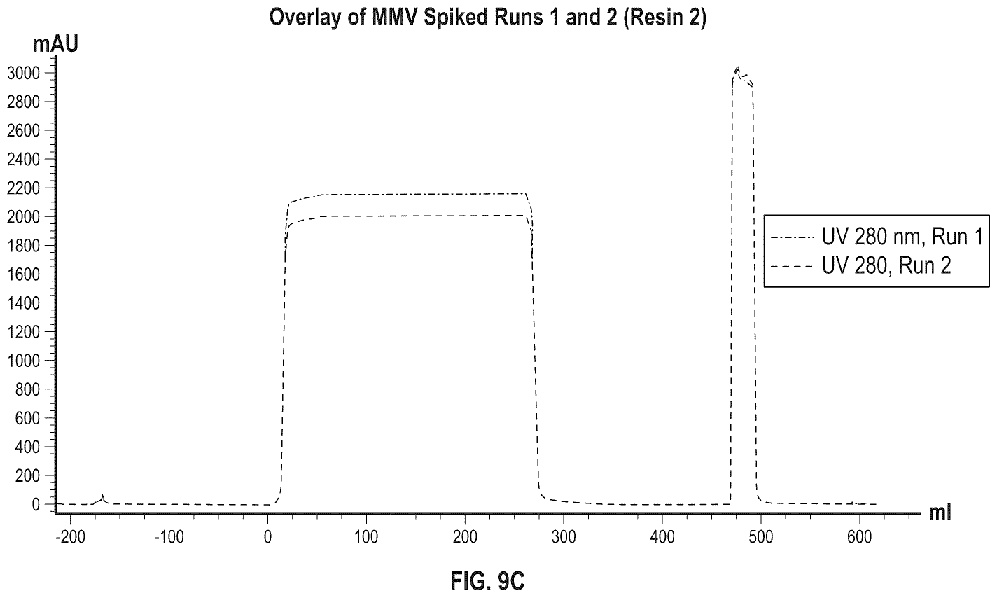

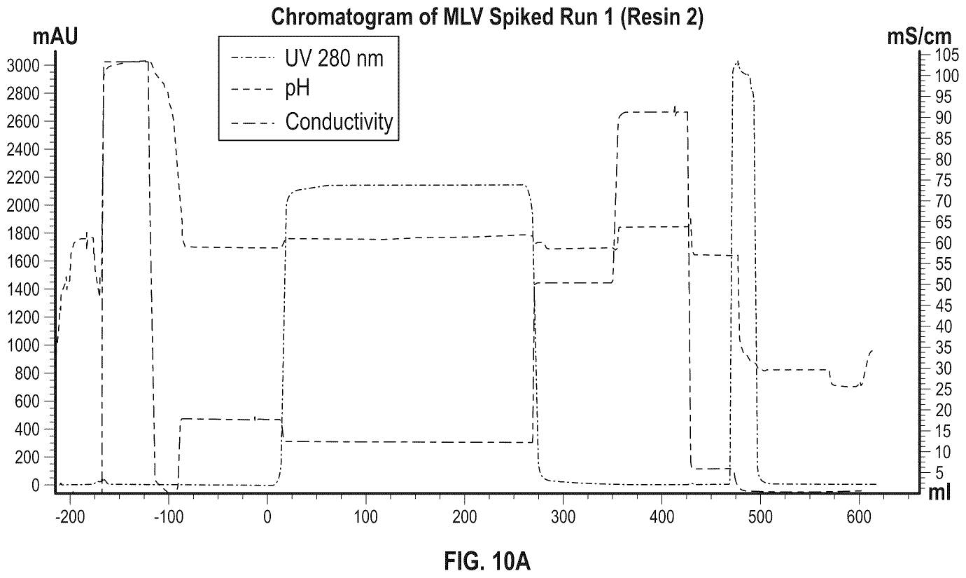

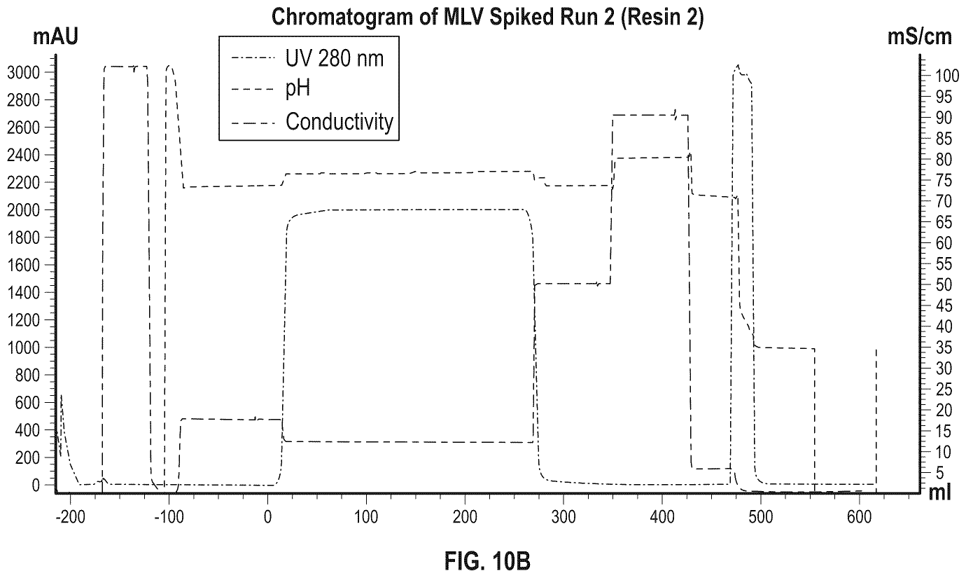

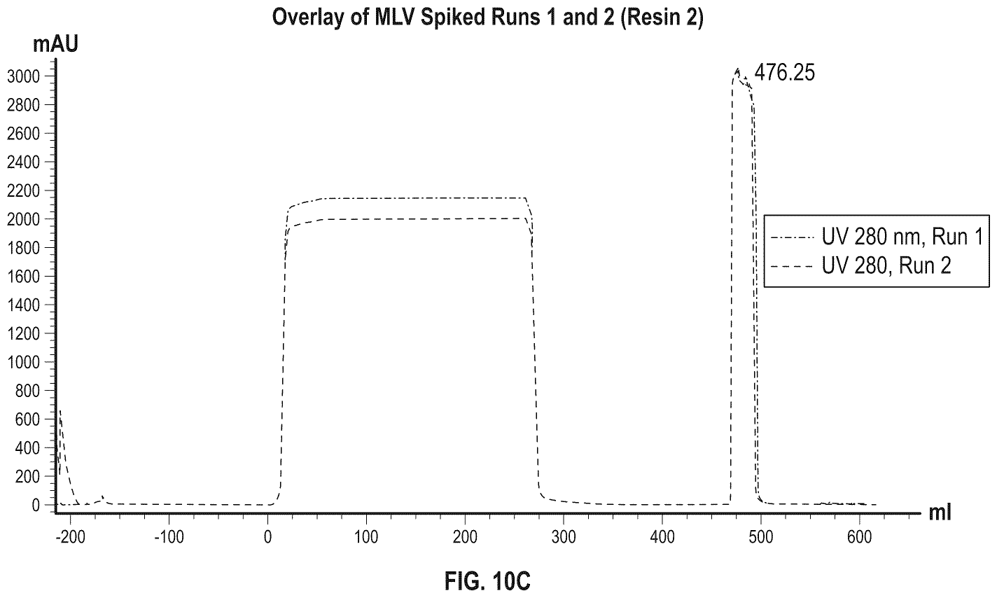

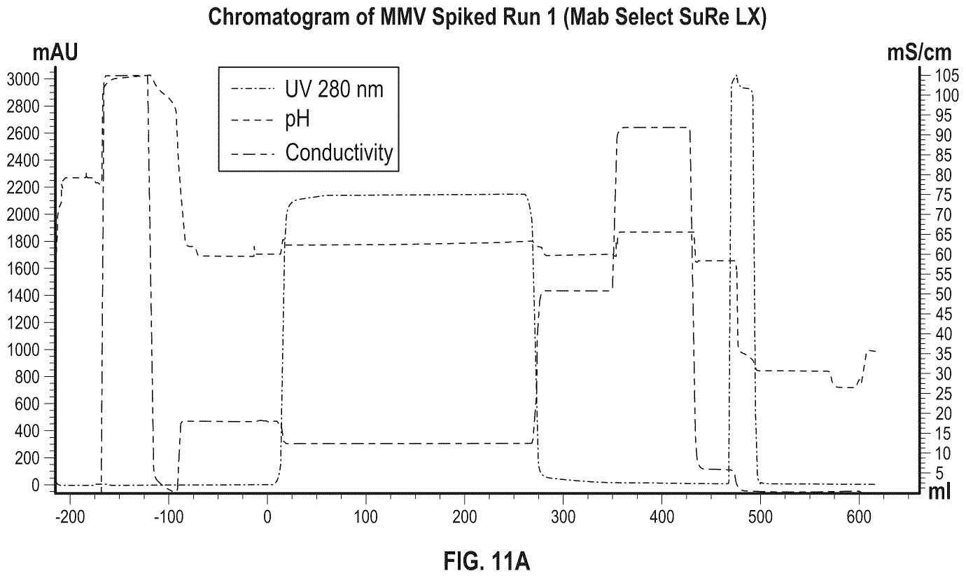

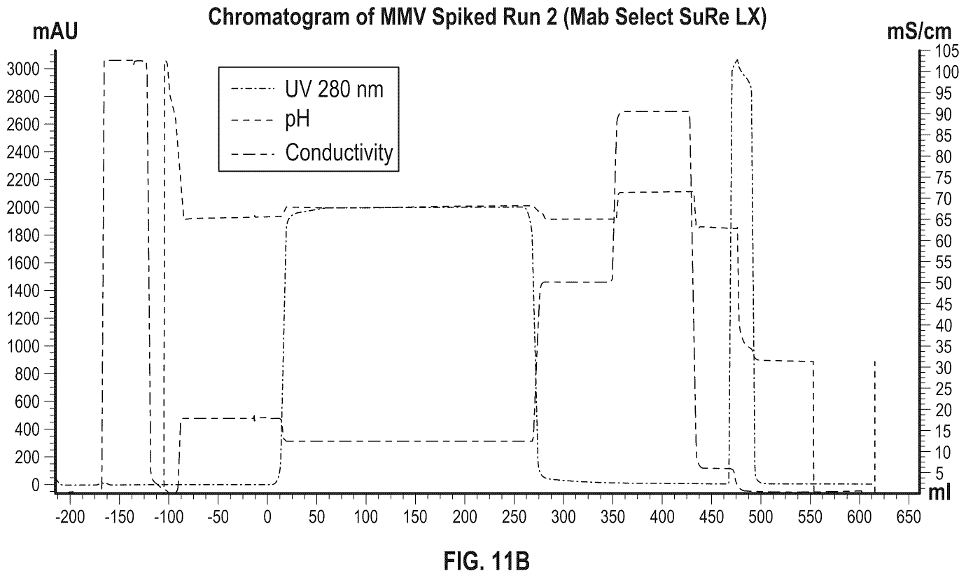

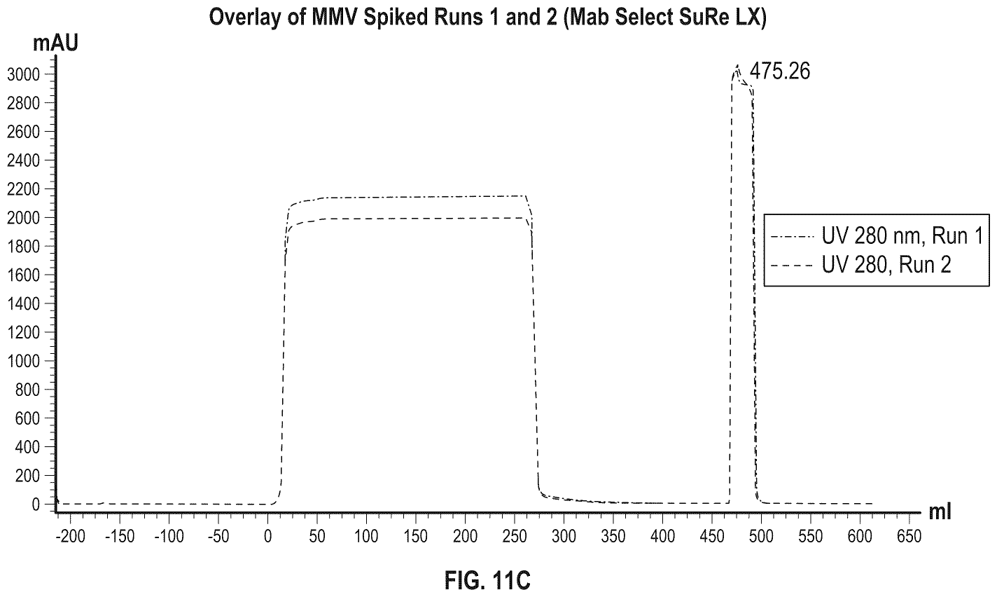

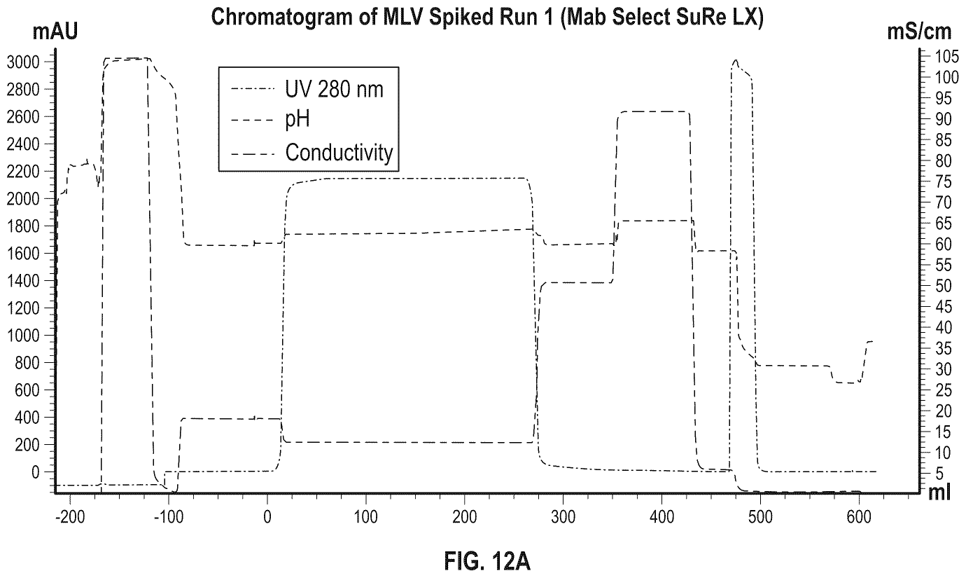

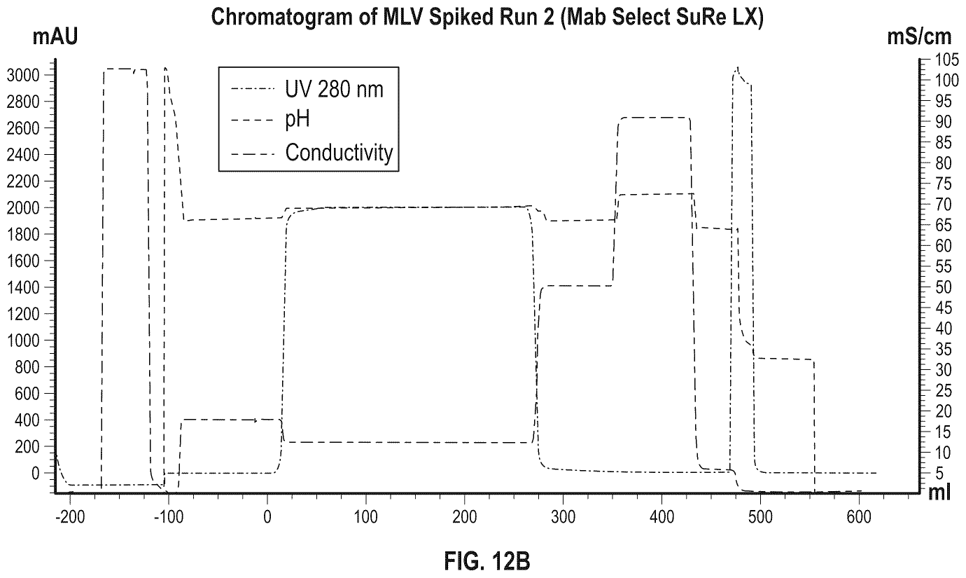

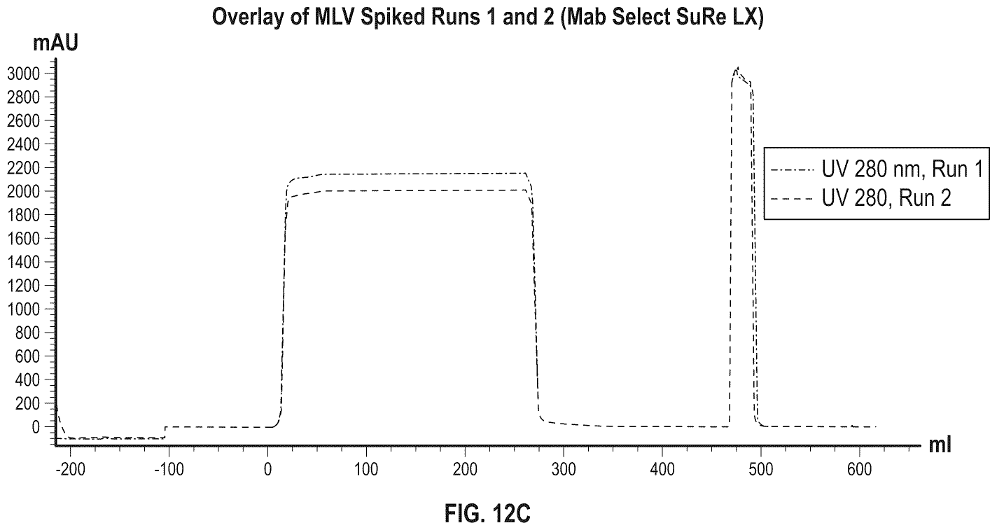

An overlay of the UV A280nm profile of the chromatograms for the virus spiked runs are shown in FIGS. 5C, 6C, 7C, 8C, 9C, 10C, 11C, and 12C.

The virus spiked liquid sample was loaded on to the equilibrated column. Following three wash steps, elution buffer was applied to the column. The UV detector was autozeroed at the end of Wash 3 for all runs performed with the Resin 1. The product eluate peak was collected from 20 mAU as the UV increased and stopped at 20 mAU on the downward slope of the peak.

Results. Virus testing results indicate significant clearance for each chromatography resin studied. MLV and MMV clearance is significant over all the Protein A chromatography resins studied as the log reduction factor is greater than or equal to 2 log₁₀. A summary of the log reduction factors of MLV and MMV for each column tested is shown in Table 13.

TABLE 13
Summary of Log Reduction Factors.
Log Reduction Factors

| | RUN 1 | RUN 2 |
|---|---|---|
| RESIN 2 AFFINITY | | |
| CHROMATOGRAPHY(compara- | | |
| tive example to Resin 1) | | |
| MLV (QPCR analysis) | 2.98 ± 0.06 log₁₀ | 2.90 ± 0.06 log₁₀ |
| MMV | 2.28 ± 1.00 log₁₀ | 2.19 ± 1.02 log₁₀ |
| RESIN 1 pH 3.5 | | |
| MLV (QPCR analysis) | 3.71 ± 0.06 log₁₀ | 3.76 ± 0.06 log₁₀ |
| MMV | 2.99 ± 0.45 log₁₀ | 2.85 ± 0.53 log₁₀ |
| RESIN 1 pH 4.5 | | |
| MLV (QPCR analysis) | 4.85 ± 0.06 log₁₀ | 5.02 ± 0.06 log₁₀ |
| MMV | 3.85 ± 0.52 log₁₀ | 4.27 ± 0.43 log₁₀ |
| Mab Select SuRe LX | | |
| AFFINITY CHROMATOG- | | |
| RAPHY | | |
| MLV (QPCR analysis) | 3.15 ± 0.06 log₁₀ | 3.09 ± 0.06 log₁₀ |
| MMV | 2.82 ± 0.42 log₁₀ | 2.58 ± 0.43 log₁₀ |

The affinity chromatography resin of Resin 1 provided more virus removal for both viruses tested as compared to the example affinity chromatography resins of Resin 2 and Mab Select SuRe LX (Cytiva). For clearance of MLV, the virus log reduction for Resin 1 with high elution buffer pH 4.5 showed markedly higher virus removal (log reduction of 4.85±0.06 log₁₀) compared to eluting the same resin with the lower pH elution buffer at pH 3.5 (log reduction of 3.71±0.06 log₁₀). For clearance of MMV, the virus log reduction for Resin 1 with high elution buffer pH 4.5 also showed markedly higher virus removal (log reduction of 3.85±0.52 log₁₀) compared to eluting the same resin with the lower pH elution buffer at pH 3.5 (log reduction of 2.85±0.53 log₁₀). For both the viruses studied, performance and results for duplicate runs per virus were within 1 log of each other, indicating consistent performance of the steps and methods.

Chromatography runs carried out during the viral clearance study gave chromatogram profiles similar to those of the small-scale model runs performed prior to the spiking study. During the study the only deviation from the chromatography protocol was the inclusion of the autozero of the UV detector in the Resin 1 runs. This has not had a significant impact on the study as the start of collection was comparable between the runs and the UV rise during the elution is sharp and therefore there would be minimal lost volume on the front side of the peak collection.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A method of viral clearance by affinity chromatography, comprising:
providing a liquid sample comprising protein and virus, wherein the protein comprises an Ig sequence;
providing an affinity chromatography resin comprising at least one Ig binding protein coupled to thereto;
contacting the affinity chromatography resin with the liquid sample under conditions that allow binding of the protein comprising the Ig sequence to the at least one Ig binding protein; and
eluting the protein from the affinity chromatography resin with an elution liquid having a pH of about 4 to about 5.5 to produce an eluted protein sample;
wherein the eluted protein sample has a reduction in virus compared to the liquid sample.

2. The method of claim 1, wherein the reduction in virus is at least about 3.8 log₁₀.

3. The method of claim 1, wherein the reduction in virus is about 3.8 log₁₀ to about 4.8 log₁₀.

4. The method of claim 1, wherein flow rate of the affinity chromatography resin is up to about 200 cm/hr.

5. The method of claim 1, wherein at least a portion of the virus in the first liquid sample remains bound to the affinity chromatography resin when the protein is eluted.

6. The method of claim 1, wherein the affinity chromatography resin comprises a cross-linked polysaccharide.

7. The method of claim 1, wherein the cross-linked polysaccharide comprises agarose.

8. The method of claim 1, wherein the cross-linked polysaccharide is a caustic resistant agarose.

9. The method of claim 1, further comprising washing the affinity chromatography resin with at least one wash buffer after the step of contacting the affinity chromatography resin with the liquid sample comprising the protein and virus and before the step of eluting the protein.

10. The method of claim 9, wherein the at least one wash buffer has a pH equal to or above about pH 7.0.

11. The method of claim 9, wherein the step of washing the affinity chromatography resin with the at least one wash buffer comprises contacting the affinity chromatography resin with a first wash buffer having a pH of about 7.4, then a second wash buffer having a pH of about 8.0, and then a third wash buffer having a pH of about 7.0.

12. The method of claim 11, wherein the first wash buffer comprises sodium phosphate and sodium chloride having a pH of about 7.4, the second wash buffer comprises Tris buffer and sodium chloride having a pH of about 8.0, and the third wash buffer comprises sodium phosphate having a pH of about 7.0.

13. The method of claim 1, wherein the elution liquid is a buffer comprising sodium acetate, citrate, and/or glycine.

14. The method of claim 1, wherein the elution liquid has a pH of about 4.5.

15. The method of claim 1, wherein the elution liquid has a pH greater than 4.5.

16. The method of claim 1, wherein the Ig binding protein comprises a cross-linked polysaccharide with a Protein A functional group.

17. The method of claim 1, wherein more than 95% of the protein comprising the Ig sequence is eluted from the affinity chromatography resin.

18. The method of claim 1, wherein the at least one Ig binding protein comprises a protein having at least 80% amino acid sequence identity to sequence SEQ ID NO: 1, wherein SEQ ID NO: 1 has at least one of the following mutations:
(a) the amino acid corresponding to one or more of position 8 is isoleucine, leucine, valine or an aromatic amino acid;
(b) the amino acid corresponding to position 8 is isoleucine or tyrosine;
(c) the amino acid corresponding to one or more of positions 10, 14, 16, 17, 18, and 28 is/are selected from the group consisting of histidine or acidic amino acids selected from aspartate or glutamate;
(d) the amino acid corresponding to position 14 is histidine;
(e) the amino acid corresponding to position 17 is histidine;
(f) the amino acid corresponding to position 16 is histidine;
(g) the amino acid corresponding to position 10 is histidine (h) the amino acid corresponding to position 10 is asparagine;
(i) the amino acid corresponding to position 8 is isoleucine, the amino acid corresponding to position 14 is histidine, and the amino acid corresponding to position 29 is lysine; and
preferably the amino acid corresponding to position 46 is cysteine;
(j) the amino acid corresponding to position 43 or 46 is cysteine; and/or
(k) the amino acid corresponding to position 29 is lysine.

19. The method of claim 18, wherein the at least one Ig binding protein comprises a protein having at least 85%, 90%, 95%, or 99% amino acid sequence identity to sequence SEQ ID NO: 1 with at least one of the mutations in (a)-(k) in claim 18.

20. The method of claim 18, wherein the at least one Ig binding protein comprises a protein having at least 80%, or at least 95%, an amino acid sequence identity to sequence of SEQ ID NO: 2.

21-48. (canceled)
